(19) United States
(12) Patent Application Publication
(10) Pub. No.: US 2022/0002395 A1
(43) Pub. Date: Jan. 6, 2022

(54) Anti-plasmodium falciparum HRP-II antibody

(71) Applicant: Fapon Biotech Inc., Shenzhen,Guangdong (CN)

(72) Inventors: Peng CUI, Dongguan, Guangdong (CN); Zhiqiang HE, Dongguan, Guangdong (CN); Yuan MENG, Dongguan, Guangdong (CN); Dongmei ZHONG, Dongguan, Guangdong (CN); Lina TANG, Dongguan, Guangdong (CN); Bi LIANG, Dongguan, Guangdong (CN); Hui YOU, Dongguan, Guangdong (CN)

(21) Appl. No.: 17/293,043

(22) PCT Filed: Oct. 15, 2019
(86) PCT No.: PCT/CN2019/111242

§ 371 Date: May 12, 2021

(30) Foreign Application Priority Data
Dec. 29, 2018 (CN) 201811654293.X

(51) Int. Cl. C07K 16/20; G01N 33/569

(52) U.S. Cl. CPC C07K 16/205; G01N 33/56905; G01N 2800/26; G01N 2333/445; C07K 2317/92

(57) ABSTRACT

Provided is an anti-Plasmodium falciparum HRP-II antibody or an antigen-binding fragment thereof. The antibody comprises a heavy chain CDR1-3 shown in SEQ ID NO: 1-3 and a light chain CDR1-3 shown in SEQ ID NO: 4-6. Also provided are an application of the antibody and a method for preparing the antibody.

### CROSS-REFERENCE TO RELATED APPLICATION

The present application claims priority to Chinese Patent Application No. 201811654293.X, filed to the China National Intellectual Property Administration on Dec. 29, 2018 and entitled “Anti-Plasmodium falciparum HRP-II antibody”, the content of which is hereby incorporated by reference in its entirety.

### TECHNICAL FIELD

The present disclosure relates to a Plasmodium antibody, an application and preparation method thereof. Specifically, the present disclosure relates to an antibody of a Plasmodium falciparum histidine-rich protein II (HRP-II), an application thereof for detecting or diagnosing malaria, and a method for preparing the antibody.

### BACKGROUND

Malaria is an insect-borne infectious disease caused by infection of a Plasmodium by bite of anopheles or transfusion of blood of a person carrying the Plasmodium. There are four main types of the Plasmodium parasitizing in a human body: Plasmodium vivax (Pv), Plasmodium falciparum (Pf), Plasmodium malariae (Pm), and Plasmodium ovale (Po). The Plasmodium falciparum is a common infectious Plasmodium (75%), and is the most harmful pathogen. It has strong infectivity, rapid proliferation, and severe symptoms. A mortality rate of the primary infection is high, deaths caused by it account for more than 95% of a total deaths of infected persons, and it mainly exists in tropical areas of Africa, South America and Asia. Plasmodium falciparum infection is a main cause of death in children under 5 years old in Africa. The Plasmodium vivax is a second most common Plasmodium (20%). Symptoms caused by infection thereof are milder, but a distribution thereof is more widespread. A population threatened by it is about 2.6 to 2.85 billion, more than 80 million cases are caused each year, and it is mainly distributed in subtropical areas of Southeast Asia, South Asia and America. The World Health Organization (WHO) recommends that all suspected malaria patients should be detected for the malaria immediately. Rapid and accurate diagnosis of the disease is essential for correctly using an antimalarial drug, avoiding generation of a drug-resistant strain, controlling deterioration of the disease, and reducing the mortality rate.

Malaria diagnosis is a key point of malaria control. Classified by a detection technology principle, existing methods for detecting the Plasmodium may be divided into four types. The first type is to directly detect the Plasmodium by using a microscope, including a thick blood membrane and a thin blood membrane, this is also a gold standard for clinical diagnosis of the malaria at present. However, it is time-consuming and labor-consuming, and requires a skilled technician and a certain experiment condition. The second type is Plasmodium nucleic acid detection, a detection target is a specific nucleotide fragment such as a Plasmodium 18S ribosomal RNA, and commonly used methods are a fluorescent Polymerase Chain Reaction (PCR) method and a Loop-mediated isothermal amplification (LAMP) technology. Although this type of the methods has high sensitivity and specificity, more complicated instruments and technical conditions are required as support, it is not suitably used as a conventional detection method in malaria-endemic areas, and is difficult to promote and apply at a grassroots level. The third type is to detect a pigment of the Plasmodium, and a flow cytometry or a mass spectrometry is usually used. This method requires a professional detection instrument, and is generally used for a laboratory research, and is not suitable for on-site detection. The fourth type is to detect the Plasmodium by an antigen-antibody immune response, there are an immunochromatographic Rapid Diagnostic Test (RDT) and an Enzyme-Linked ImmunoSorbent Assay (ELISA) methodologically, and most of target antigens detected are diagnostic antigens such as LDH and HRP-II. The RDT using the antigen as a detection target has important significance while being applied in backward areas where the Plasmodium falciparum is prevalent. It is recommended by the WHO for on-site diagnosis due to advantages of its simple operation, rapidness, intuitive results, no complicated device, high sensitivity and specificity and the like.

At present, the commonly used antigens for detecting the malaria are a Histidine-rich protein II (HRP-II) unique to the Plasmodium falciparum and a Plasmodium Lactate dehydiogenase (PLDH). The HRP-II is 35-105 kDa of a water-soluble protein unique to the Plasmodium falciparum, and it is only synthesized in asexual parasite stage and early gametophyte stage of an erythrocytic stage. The synthesis is started from an immature ring body, the content is gradually increased along with cleavage of merozoites during the whole erythrocytic stage, it may last for 28 d, and it is difficult to detect in a mature gametophyte stage. The HRP-II protein is secreted into blood of a host in a soluble form. At the same time, the protein may be detected in cerebrospinal fluid, urine and saliva, and is the most recognized diagnostic marker for the Plasmodium falciparum. Detection of the HRP-II by a specific monoclonal antibody may determine the presence of the Plasmodium falciparum in a human body.

At present, there are many kits on the market that use the HRP-II as a target protein to detect the Plasmodium, such as a foreign ParaSight-F kit, an ICT Malaria P.f.&P.f/P.v diagnostic kit, a BinaxNOW® kit, and a CareStart Malaria HRP-II/PLDH composite test kit; and domestically, there are an Aikang P.f./P.v colloidal gold immunochromatographic detection kit and the like. The ParaSight-F kit is an application product recommended by the World Health Organization. A mouse anti-HRP-II IgG1 monoclonal antibody and a control HRP-II antigen which are coated on a strip-shaped nitrocellulose membrane in a linear and dotted shape are respectively used as a capture antibody and a detection line, thiorhodamine B is used as an indicator for color developing, and it may only detect falciparum malaria. The ICT Malaria P.f./P.v diagnostic kit is coated on a nitrocellulose membrane with a strain of an anti-Plasmodium falciparum HRP-II monoclonal antibody and a strain of a Plasmodium vivax specific monoclonal antibody as capture antibodies, a colloidal gold labeling is used for color developing, and it is used for falciparum malaria, vivax malaria and mixed infection. In the BinaxNOW® kit, 2 different monoclonal antibodies are applied, detection target proteins are HRP-II and aldolase, the HRP-II is unique to the falciparum malaria, the aldolase is a component shared by 4 types of the Plasmodium infecting the human body, and the simple falciparum malaria and other human Plasmodium except the falciparum malaria are simultaneously detected. The CareStart Malaria HRP-II/PLDH composite detection kit uses 2 monoclonal monomers to form two independent detection lines on a membrane, they are respectively an anti-Plasmodium (Falciparum Plasmodium, Plasmodium vivax, Plasmodium ovale, and Plasmodium vivax) lactate dehydrogenase (PLDH) monoclonal antibody and an anti-HRP-II monoclonal antibody, and specifically used for the differential diagnosis of falciparum malaria and other types of the malaria. The Aikang P.f/P.v colloidal gold immunochromatographic detection kit respectively uses anti-Plasmodium falciparum HRP-II and anti-Plasmodium vivax PLDH monoclonal antibodies as capture antibodies, and the infections of the simple Plasmodium falciparum and simple Plasmodium vivax are diagnosed simultaneously.

In the above kits, the anti-HRP-II monoclonal antibodies are used. At present, a conventional preparation method of the monoclonal antibodies used for diagnosis in the market is a hybridoma technology, namely a genetic engineering technology is used to express the HRP-II protein to immunize mice, immunized mouse spleen cells are fused with tumor cells to obtain hybridoma cells, and finally the hybridoma cells secreting the target antibody are screened out, and then the antibody production is performed. However, in a traditional hybridoma technology, during a process of culture or cryopreservation and recovery of the hybridoma cells, some cells may lose an ability to secrete the antibodies, so that some precious cell strains are lost. In addition, while a large number of the antibodies are generated, a large number of the hybridoma cells are cultured in vitro, a yield thereof is low, and a production cost is high; during a process of mouse abdominal cavity induction, due to the influence of a size of an individual mouse, the generation of the antibodies is unstable, a difference between batches is large, and purification is difficult because mouse autoantibodies are contained.

In order to avoid disadvantages of the traditional hybridoma technology, the present disclosure designs an expression vector of an anti-HRP-II monoclonal antibody, and provides an anti-HRP-II monoclonal antibody sequence with high activity and high affinity, a host cell for expressing an anti-HRP-II monoclonal antibody through a recombinant technology, and a method for malaria diagnosis with high specificity and sensitivity.

### SUMMARY

The present disclosure provides an anti-Plasmodium falciparum HRP-II antibody or an antigen-binding fragment thereof. The present disclosure further provides a preparation method for the antibody, an antibody conjugate, a fusion protein and a kit/diagnostic agent containing the antibody, and an application of the antibody for diagnosing a Plasmodium falciparum infection.

In one or more implementation schemes, the present disclosure provides an antibody or an antigen-binding fragment thereof, the antibody binds to a Plasmodium falciparum histidine-rich protein-II, namely HRP-II, herein the antibody includes complementarity determining regions having the following amino acid sequences or at least 80% sequence identity (for example, at least 81%, 82%, 83%, 84%, 85%, 86%, 87%, 88%, 89%, 90%, 91%, 92%, 93%, 94%, 95%, 96%, 97%, 98%, 99% or higher sequence identity) with thereof:

a heavy chain CDR1, herein it includes or consists of an amino acid sequence G-X1-T-X2-T-X3-Y-Y-M-N shown in SEQ ID NO:1, herein X1 is Y, T or S; X2 is L or I; and X3 is D or E, preferably D;

a heavy chain CDR2, herein it includes or consists of an amino acid sequence D-X1-N-P-I-X2-G-G-T-X3-Y-X4-Q-K-F shown in SEQ ID NO: 2, herein X1 is L, V or I; X2 is Q or N; X3 is A or P, preferably A; and X4 is Q or N; and

a heavy chain CDR3, herein it includes or consists of an amino acid sequence T-X1-G-A-E-X2-Y shown in SEQ ID NO: 3, wherein X1 is K or R; and X2 is D or E, preferably D; and the antibody further includes:

a light chain CDR1, herein it includes or consists of an amino acid sequence S-Q-S-X1-Y-S-X2-G-K-1-Y-X3-N shown in SEQ ID NO: 4, herein X1 is LL, IL, II or LI; X2 is Q or N; and X3 is I or L, preferably L;

a light chain CDR2, herein it includes or consists of an amino acid sequence Q-X1-S-K-X2-X3-P shown in SEQ ID NO: 5, herein X3 is I, V or L; X2 is I or L, preferably L; and X3 is D or E; and

a light chain CDR3, herein it includes or consists of an sequence L-Q-X1-T-Y-X2-P-X3-T shown in SEQ ID NO: 6, herein X1 is A or G; X2 is 3, Y or T; and X3 is Q or N, preferably Q;

for example, the antibody may include the heavy chain CDR1, the heavy chain CDR2, the heavy chain CDR3, the light chain CDR1, the light chain CDR2, and the light chain CDR3 containing amino acid residue substitution combinations as shown in the following table:

i) the antibody contains the amino acid residue substitution combinations shown in the table below:

| | CDR-VH1 | CDR-VH2 | CDR-VH3 | CDR-VL1 | CDR-VL2 | CDR-VL3 |
|---|---|---|---|---|---|---|
| Site | X3 | X3 | X2 | X3 | X2 | X3 |
| Mutation 1 | D | A | D | L | L | Q |

ii) the antibody in the i) further contains the amino acid residue substitution combinations shown in the table below:

| | CDR-VH1 | CDR-VH2 | CDR-VH3 | CDR-VL1 | CDR-VL2 | CDR-VL3 |
|---|---|---|---|---|---|---|
| Site | X1/X2 | X1/X2/X4 | X1 | X1/X2 | X1/X3 | X1/X2 |
| Mutation 1-0 | Y/L | L/Q/N | K | LL/Q | I/D | A/S |
| Mutation 1-1 | Y/I | I/Q/N | R | II/Q | I/E | G/Y |
| Mutation 1-2 | S/L | V/Q/Q | K | LL/N | L/D | A/T |
| Mutation 1-3 | Y/I | L/N/Q | R | II/N | L/E | G/S |
| Mutation 1-4 | Y/L | I/N/Q | K | IL/N | V/D | A/Y |
| Mutation 1-5 | S/L | V/N/N | R | IL/Q | V/E | G/T |
| Mutation 1-6 | Y/I | L/Q/N | K | LI/N | I/D | A/S |
| Mutation 1-7 | Y/I | I/N/Q | R | LI/N | I/E | A/Y |
| Mutation 1-8 | Y/I | V/Q/N | K | LL/Q | L/D | A/T |
| Mutation 1-9 | Y/L | L/Q/N | R | II/Q | L/E | G/S |
| Mutation 1-10 | Y/I | I/N/N | R | LL/N | V/D | G/Y |
| Mutation 1-11 | Y/I | V/N/N | K | II/N | V/E | A/T |
| Mutation 1-12 | Y/I | L/N/N | R | IL/N | I/D | G/S |
| Mutation 1-13 | S/L | I/Q/N | K | IL/Q | I/E | G/Y |
| Mutation 1-14 | Y/L | V/N/N | K | LI/N | L/D | A/T |
| Mutation 1-15 | Y/I | L/Q/N | R | LI/N | L/E | G/T |
| Mutation 1-16 | Y/I | I/Q/N | R | LL/Q | V/D | G/S |
| Mutation 1-17 | Y/I | V/N/N | K | LL/Q | V/E | G/Y |
| Mutation 1-18 | Y/L | L/Q/N | K | LL/Q | I/D | G/T |
| Mutation 1-19 | T/L | I/Q/N | R | II/Q | I/E | A/S |
| Mutation 1-20 | S/I | V/Q/N | K | II/Q | L/D | G/Y |
| Mutation 1-21 | T/L | L/Q/N | R | LL/Q | L/E | G/T |
| Mutation 1-22 | Y/I | I/Q/Q | K | II/N | V/D | A/S |
| Mutation 1-23 | T/L | V/Q/N | R | II/N | V/E | G/Y |
| Mutation 1-24 | T/L | L/Q/Q | K | II/N | I/D | A/S |
| Mutation 1-25 | Y/I | I/Q/N | R | LL/Q | I/E | G/T |
| Mutation 1-26 | T/L | V/Q/N | R | LL/Q | L/D | G/Y |
| Mutation 1-27 | T/L | L/N/Q | K | II/N | L/E | G/S |
| Mutation 1-28 | Y/I | I/Q/Q | K | II/N | V/D | A/T |
| Mutation 1-29 | Y/I | V/Q/N | R | II/Q | V/E | G/Y |
| Mutation 1-30 | S/L | L/Q/N | K | II/N | I/D | A/S |
| Mutation 1-31 | T/L | I/N/Q | K | LL/N | I/E | G/T |
| Mutation 1-32 | Y/I | V/Q/Q | R | LL/N | L/D | A/Y |
| Mutation 1-33 | T/L | L/Q/N | K | LL/N | L/E | G/S |
| Mutation 1-34 | T/L | I/N/Q | R | LL/N | V/D | G/T |
| Mutation 1-35 | Y/I | V/Q/Q | R | LL/N | V/E | A/Y |
| Mutation 1-36 | T/L | L/Q/N | R | LL/N | I/D | G/S |
| Mutation 1-37 | Y/I | I/N/Q | K | LL/N | I/E | A/Y |
| Mutation 1-38 | T/L | V/Q/Q | R | LL/N | L/D | G/S |
| Mutation 1-39 | T/L | L/N/Q | K | LL/N | L/E | A/T |
| Mutation 1-40 | T/L | I/N/Q | R | IL/N | L/D | G/T |
| Mutation 1-41 | Y/L | V/N/Q | K | IL/N | V/E | A/Y |
| Mutation 1-42 | Y/I | L/N/Q | R | IL/N | I/D | G/S |
| Mutation 1-43 | T/L | I/N/Q | K | IL/N | I/E | G/S |
| Mutation 1-44 | Y/I | V/N/Q | R | IL/N | L/D | A/S |
| Mutation 1-45 | Y/I | L/N/N | K | IL/Q | L/E | G/Y |
| Mutation 1-46 | Y/I | I/N/N | R | IL/Q | V/D | G/T |
| Mutation 1-47 | S/I | V/N/N | K | IL/Q | V/E | A/S |
| Mutation 1-48 | Y/I | L/Q/N | R | LL/Q | I/D | G/Y |
| Mutation 1-49 | Y/L | I/Q/N | K | LL/Q | I/E | A/T |
| Mutation 1-50 | Y/I | V/Q/Q | R | LL/N | L/E | A/Y |
| Mutation 1-51 | S/L | L/N/Q | K | LL/N | L/E | G/Y |
| Mutation 1-52 | T/L | I/Q/Q | R | II/N | V/D | A/Y |

herein the antibody binds to the HRP-II protein in a KID of 10⁻⁹M or less.

In one or more implementation schemes, the present disclosure provides an antibody or an antigen-binding fragment thereof, the antibody binds to the HRP-II, herein the antibody includes:

a heavy chain CDR1, herein it includes or consists of an amino acid sequence shown in SEQ ID NO: 21;

a heavy chain CDR2, herein it includes or consists of an amino acid sequence shown in SEQ ID NO: 22; and

a heavy chain CDR3, herein it includes or consists of an amino acid sequence shown in SEQ ID NO: 23; and

the antibody further includes:

a light chain CDR1, herein it includes or consists of an amino acid sequence shown in SEQ ID NO: 24;

a light chain CDR2, herein it includes or consists of an amino acid sequence shown in SEQ ID NO: 25; and

a light chain CDR3, herein it includes or consists of an sequence shown in SEQ ID NO: 26.

In one or more implementation schemes, the antibody of the present disclosure further includes framework regions of a heavy chain variable region FR-H1, FR-H2, FR-H3 and FR-H4 and framework regions of a light chain variable region FR-L1, FR-L2, FR-L3 and FR-L4,

herein the FR-H1 includes or consists of an amino acid sequence of SEQ ID NO: 27, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 27, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 27;

the FR-H2 includes or consists of an amino acid sequence of SEQ ID NO: 28, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 28, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 28;

the FR-H3 includes or consists of an amino acid sequence of SEQ ID NO: 29, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 29, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 29;

the FR-H4 includes or consists of an amino acid sequence of SEQ ID NO: 30, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 30, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 30;

the FR-L1 includes or consists of an amino acid sequence of SEQ ID NO: 31, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 31, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 31;

the FR-L2 includes or consists of an amino acid sequence of SEQ ID NO: 32, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 32, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 32;

the FR-L3 includes or consists of an amino acid sequence of SEQ ID NO: 33, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 33, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 33; and

the FR-L4 includes or consists of an amino acid sequence of SEQ ID NO: 34, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 34, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 34.

In one or more implementation schemes, the present disclosure provides an antibody or an antigen-binding fragment thereof, the antibody binds to the HRP-II, herein the antibody includes:

(i) a heavy chain variable region, herein it includes or consists of the following sequences:

an amino acid sequence shown in SEQ ID NO: 17, or

a sequence having at least 70%, 75%, 80%, 85%, and 90%, preferably at least 91%, 92%, 93%, 94%, 95%, 96%, 97%, 98%, 99% or 100%, of sequence identity with the sequence shown in the SEQ ID NO: 17, or

an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 17; and

(II) a light chain variable region, herein it includes or consists of the following sequences:

an amino acid sequence shown in SEQ ID NO: 19, or

a sequence having at least 70%, 75%, 80%, 85%, and 90%, preferably at least 91%, 92%, 93%, 94%, 95%, 96%, 97%, 98%, 99% or 100%, of sequence identity with the sequence shown in the SEQ ID NO: 19, or

an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 19.

In one or more implementation schemes, the antibody of the present disclosure binds to the HRP-II protein in a KD of 10-10 M or less.

In one or more implementation schemes, the antibody of the present disclosure binds to the HRP-II protein in an EC50 of less than about 100 nM, for example, less than about 10 nM, 1 nM, 0.9 nM, 0.8 nM, 0.7 nM, 0.6 nM, 0.5 nM, 0.4 nM, 0.3 nM, 0.2 nM, 0.1 nM or less.

In one or more implementation schemes, the antigen-binding fragment of the antibody of the present disclosure may include Fab, Fab′, F(ab′)2, Fd, Fv, a complementarity determining region (CDR) fragment, a single-chain antibody (for example, scFv), a bivalent antibody or a domain antibody.

In one or more implementation schemes, the present disclosure provides an isolated polypeptide, it is selected from a group formed by the following items:

(1) an isolated polypeptide, herein it includes sequences shown in SEQ ID NO: 21, 22 and 23, herein the polypeptide is served as a part of an anti-HRP-II antibody, and specifically binds to the HRP-II, and the antibody further includes sequences shown in SEQ ID NO: 24, 25 and 26;

(2) an isolated polypeptide, herein it includes sequences shown in SEQ ID NO: 24, 25 and 26, herein the polypeptide is served as a part of the anti-HRP-II antibody, and specifically binds to the HRP-II, and the antibody further includes sequences shown in SEQ ID NOs: 21, 22 and 23;

(3) an isolated polypeptide, herein it includes the sequence shown in the SEQ ID NO: 17, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, herein the polypeptide is served as a part of the anti-HRP-II antibody, and specifically binds to the HRP-II, and the antibody further includes the sequence shown in the SEQ ID NO: 19, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence;

(4) an isolated polypeptide, herein it includes the sequence shown in the SEQ ID NO: 19, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, herein the polypeptide is served as a part of the anti-HRP-II antibody, and specifically binds to the HRP-II, and the antibody further includes the sequence shown in the SEQ ID NO: 17, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence;

(5) an isolated polypeptide, herein it includes the sequence shown in the SEQ ID NO: 18, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, herein the polypeptide is served as a part of the anti-HRP-II antibody, and specifically binds to the HRP-II, and the antibody further includes the sequence shown in the SEQ ID NO: 20, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence; and

(6) an isolated polypeptide, herein it includes the sequence shown in the SEQ ID NO: 20, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, herein the polypeptide is served as a part of the anti-HRP-II antibody, and specifically binds to the HRP-II, and the antibody further includes the sequence shown in the SEQ ID NO: 18, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence.

In one or more implementation schemes, the present disclosure provides an isolated polynucleotide, it encodes the antibody or the antigen-binding fragment thereof or the separated polypeptide of the present disclosure.

In one or more implementation schemes, the present disclosure provides a vector, herein it includes the separated polynucleotide described in this article.

In one or more implementation schemes, the present disclosure provides a host cell, herein it includes the separated polynucleotide or the vector described in this article.

In one or more implementation schemes, the present disclosure provides a method for preparing the antibody or the antigen-binding fragment thereof described in this article, herein the method includes culturing the host cell described in this article.

In one or more implementation schemes, the present disclosure provides an antibody conjugate, herein it includes the antibody or the antigen-binding fragment thereof described in this article and a coupling portion coupled with the same, preferably, the coupling portion includes a purification tag (such as a His tag), a detectable label, such as a colloidal gold, a radioactive label, a luminescent substance, a colored substance, an enzyme, such as a fluorescent label, a chromophore label, an electronic dense label, such as a radioisotope, a fluorophore, rhodamine and a derivative thereof, a luciferase, a luciferin, a horseradish peroxidase, an alkaline phosphatase, a P-galactosidase, a glucoamylase, a lysozyme, a carbohydrate oxidase, a glucose oxidase, a galactose oxidase, and a glucose-6-phosphate dehydrogenase, biotin/avidin, and a spin label.

In one or more implementation schemes, the present disclosure provides a kit or a diagnostic agent, herein it includes the antibody or the antigen-binding fragment thereof described in this article, an antibody conjugate or a fusion protein, herein:

1) preferably, the kit further includes an antibody which binds to a Plasmodium antigen except the HRP-II or an antigen-binding fragment thereof, an antibody conjugate, or a fusion protein, for example, Plasmodium falciparum, Plasmodium vivax, Plasmodium malariae and/or Plasmodium ovale specific antigens or shared antigens, such as an aldolase, such as a lactate dehydrogenase PLDH, such as a Plasmodium lactate dehydrogenase PLDH or a Plasmodium species-specific lactate dehydrogenase PLDH;

2) preferably, the kit further includes another one or more antibodies, herein it specifically recognizes the antibody or the antigen-binding fragment described in this article, the antibody conjugate, or the fusion protein; and/or specifically recognizes the antibody which binds to the Plasmodium antigen except the HRP-II or the antigen-binding fragment thereof, the antibody conjugate, or the fusion protein. Optionally, the another one or more antibodies further include a detectable label, such as a colloidal gold, a radioactive label, a luminescent substance, a colored substance, an enzyme, such as a fluorescent label, a chromophore label, an electronic dense label, such as a radioisotope, a fluorophore, rhodamine and a derivative thereof, a luciferase, a luciferin, a horseradish peroxidase, an alkaline phosphatase, a β-galactosidase, a glucoamylase, a lysozyme, a carbohydrate oxidase, a glucose oxidase, a galactose oxidase, and a glucose-6-phosphate dehydrogenase, biotin/avidin, and a spin label;

3) optionally, the kit is a Rapid Diagnostic Test (RDT) kit, such as a kit for applying thin film immunochromatography, for example, a kit for thin film immunochromatography using a colloidal gold method, for example, a solid-phase support such as a membrane support is contained, the antibody which binds to the Plasmodium antigen or the antigen-binding fragment thereof, the antibody conjugate, or the fusion protein and an optional control antibody are immobilized to the solid-phase support; and

4) optionally, the kit is an Enzyme-Linked ImmunoSorbent Assay (ELISA) kit.

In one or more implementation schemes, the present disclosure provides an application of the antibody or the antigen-binding fragment thereof, the antibody conjugate, or the fusion protein described in this article in preparing a kit or a diagnostic agent, the kit is used for 1) detecting the presence of the HRP-II in a sample or a level thereof, 2) diagnosing a Plasmodium falciparum infection, and/or 3) identifying and diagnosing the Plasmodium falciparum infection and other Plasmodium infections.

In one or more implementation schemes, a source of the sample is not particularly limited, for example, it may include a tissue, a cell or a fluid sample, such as a sample of a body fluid, such as a cerebrospinal fluid, a urine, a saliva, or a blood sample. In order to avoid disadvantages of the traditional hybridoma technology, the present disclosure provides an expression vector of an anti-Plasmodium falciparum HRP-II monoclonal antibody, and provides an anti-Plasmodium falciparum HRP-II monoclonal antibody sequence, it is used for expressing the anti-Plasmodium falciparum HRP-II monoclonal antibody through a recombinant technology, and used for diagnosis of falciparum malaria.

In one or more implementation schemes, the present disclosure includes one or more of the following aspects:

1) cloning of a light chain gene of the anti-HRP-II monoclonal antibody;

2) cloning of a heavy chain gene of the anti-HRP-II monoclonal antibody;

3) construction of an expression vector of the anti-HRP-II monoclonal antibody;

4) for the expression vector of the step 3), an expression system, for example a eukaryotic expression system such as a CHO eukaryotic expression system, is used to obtain the Anti-HRP-II monoclonal antibody; and

5) for the step 4), activity identification and affinity analysis are performed on the Anti-HRP-II monoclonal antibody.

### BRIEF DESCRIPTION OF THE DRAWINGS

FIG. 1: a purification antibody reducibility SDS-PAGE diagram, herein lanes 1 and 2 show 5 μg antibody reducibility SDS-PAGE diagrams obtained by purification of a Protein A affinity chromatography column.

### DETAILED DESCRIPTION OF THE EMBODIMENTS

Specific implementation modes of the present disclosure are described in detail below, and each specific implementation mode described is not restrictive, and may be combined with each other.

A term “amino acid” refers to a naturally existing or non-naturally existing carboxy a-amino acid.

The term “amino acid” used in the present application may include a naturally existing amino acid and a non-naturally existing amino acid. The naturally existing amino acid includes an alanine (three-letter code: Ala, one-letter code: A), an arginine (Arg, R), an asparagine (Asn, N), an aspartic acid (Asp, D), a cysteine (Cys, C), a glutamine (Gin, Q), a glutamic acid (Glu, E), a glycine (Gly, G), a histidine (His, H), an isoleucine (Ile, I), a leucine (Leu, L), a lysine (Lys, K), a methionine (Met, M), a phenylalanine (Phe, F), a proline (Pro, P), a serine (Ser, S), a threonine (Thr, T), a tryptophan (Trp, W), a tyrosine (Tyr, Y), and a valine (Val, V). The non-naturally existing amino acid includes but is not limited to an a-aminoadipate, an aminobutyric acid, a citrulline, a homocitrulline, a homoleucine, a homoarginine, a hydroxyproline, a norleucine, a pyridine alanine, a sarcosine and the like.

In this article, a peptide, a polypeptide, and a protein are not strictly distinguished, and may be used interchangeably in one or more situations. Generally, it refers to a polymer formed by the amino acid connected by a peptide bond whether it is naturally generated or synthesized. The polypeptide may also contain a non-amino acid component, such as a carbohydrate group, a metal ion, or a carboxylic acid ester. The non-amino acid component may be added by a cell for expressing the polypeptide, and may be different along with cell types. The polypeptide is defined in this article With regard to an amino acid framework structure thereof or a nucleic acid encoding it. For example, the addition of the carbohydrate group is usually not specified, but it may be existent. All polypeptide sequences are written in accordance with generally accepted conventions, herein an α-N-terminal amino acid residue is on the left, and an α-C-terminal amino acid residue is on the right. As used herein, a term “N-terminal” refers to a free α-amino group of the amino acid in the polypeptide, and a term “C-terminal” refers to a free α-carboxylic acid terminal of the amino acid in the polypeptide. The polypeptide terminated with a certain group at the N-terminal refers to a polypeptide that carries a group on a-amino nitrogen of the N-terminal amino acid residue. The amino acid terminated with a certain group at the N-terminal refers to an amino acid that carries a group on a-amino nitrogen.

“Conservative” amino acid substitutions are those substitutions in which the amino acid residue is substituted by an amino acid residue with a side chain having similar physicochemical properties. The amino acid residues with similar side chains are known in the field, and include an amino acid with a basic side chain (for example, a lysine, an arginine, and a histidine), an amino acid with an acidic side chain (for example, an aspartic acid, and a glutamic acid), an amino acid with an uncharged polar side chain (for example, a glycine, an asparagine, a glutamine, a serine, a threonine, a tyrosine, a cysteine, and a tryptophan), an amino acid with a non-polar side chain (for example, an alanine, a valine, a leucine, an isoleucine, a proline, a phenylalanine, and a methionine), an amino acid with a P-branched side chain (for example, a threonine, a valine, and an isoleucine) and an amino acid with an aromatic side chain (for example, a tyrosine, a phenylalanine, a tryptophan, and a histidine). Special forms of the conservative amino acid substitutions include those amino acid substitutions which are not used in 20 normal amino acids encoded by a genetic code. The implementation scheme of the present disclosure includes the use of a synthetic peptide, so such the “non-naturally existing” amino acid residue may be used in the peptide disclosed in this article, and a natural saturated carbon chain in the side chain of the amino acid residue may be exchanged by a shorter or longer saturated carbon chain.

In the case of two amino acid sequences, while comparison is optimized, such as while default gap weight comparison is used through a procedure GAP or BESTFIT, the sequence of the present disclosure contains a corresponding sequence having at least 99%, 98%, 97%, 96%, 95%, 94%, 93%, 92%, 91%, 90%, 89%, 88%, 87%, 86%, 85%, 84%, 83%, 82%, 81%, 80%, 75%, and 70% of sequence identity with sequences shown in a sequence table. For example, in one or more implementation schemes, the antibody of the present disclosure includes antibodies of corresponding heavy chains CDR and light chains CDR having at least 99%, 98%, 97%, 96%, 95%, 94%, 93%, 92%, 91%, 90%, 89%, 88%, 87%, 86%, 85%, 84%, 83%, 82%, 81%, 80%, 75%, and 70% of the sequence identity with heavy chains CDR shown in SEQ ID NO: 1-3, and light chains CDR shown in SEQ ID NO: 4-6, or heavy chains CDR shown in SEQ ID NO: 21-23, and light chains CDR shown in SEQ ID NO: 24-26. For example, in one or more implementation schemes, the antibody of the present disclosure includes antibodies of corresponding heavy chains FR1-4 and light chains FR1-4 having at least 99%, 98%, 97%, 96%, 95%, 94%, 93%, 92%, 91%, 90%, 89%, 88%, 87%, 86%, 85%, 84%, 83%, 82%, 81%, 80%, 75%, and 70% of the sequence identity with heavy chains FR1-4 shown in SEQ ID NO: 27-30 and light chains FR1-4 shown in SEQ ID NO: 31-34. For example, in one or more implementation schemes, the antibody of the present disclosure includes antibodies of corresponding heavy chain variable region and light chain variable region having at least 99%, 98%, 97%, 96%, 95%, 94%, 93%, 92%, 91%, 90%, 89%, 88%, 87%, 86%, 85%, 84%, 83%, 82%, 81%, 80%, 75%, and 70% of the sequence identity with a heavy chain variable region shown in SEQ ID NO: 17, and a light chain variable region shown in SEQ ID NO: 19. For example, in one or more implementation schemes, the antibody of the present disclosure includes antibodies of corresponding heavy chain and light chain having at least 99%, 98%, 97%, 96%, 95%, 94%, 93%, 92%, 91%, 90%, 89%, 88%, 87%, 86%, 85%, 84%, 83%, 82%, 81%, 80%, 75%, and 70% of the sequence identity with a heavy chain shown in SEQ ID NO: 18, and a light chain variable region shown in SEQ ID NO: 20, In one or more implementation schemes, the present disclosure contains an antigen-binding fragment of the antibody, an antibody conjugate, or a fusion protein. In one or more implementation schemes, the present disclosure further includes a corresponding separated polypeptide containing the above antibody heavy chains and/or light chains. In one or more implementation schemes, the present disclosure includes a polynucleotide, a vector, and a host cell for encoding the antibody and separated polypeptide.

In one or more implementation schemes, a difference between the different residues of the antibody of the present disclosure is the conservative amino acid substitution. As known to those skilled in the art, sequence analysis software may be used to measure the sequence identity. For example, publicly available GCG software includes programs such as “Gap” and “BestFit”, the software may be used with default parameters to determine sequence homology or sequence identity between closely related polypeptides or between a wild-type protein and a mutant protein thereof. FASTA or ClustalW may also be used, and default or recommended parameters are used to compare the peptide sequences. The program in GCG version 6.1, FASTA (for example, FASTA2 and FASTA3), gives the comparison and percent sequence identity of an optimal overlap region between query and search sequences. Another algorithm is a computer program BLAST which uses the default parameters, especially blastp.

A term “antibody” in this article is used in the broadest sense, and it may include a full-length monoclonal antibody, a bispecific or multispecific antibody, a chimeric antibody, and an antibody fragment, as long as they show the desired biological activity, such as specifically binding to the HRP-II antigen or the fragment thereof. The “antibody fragment” includes a portion of the full-length antibody, preferably the antigen-binding region or the variable region thereof. Examples of the antibody fragment include Fab, Fab′, F(ab′)2, Fd, Fv, a complementarity determining region (CDR) fragment, a single chain antibody (for example, scFv), a bivalent antibody or a domain antibody. In one or more implementation schemes, the present disclosure includes a fusion protein of the antibody or the antibody fragment, for example, it is fused with a detectable label or tag, such as GFP, HA, Flag, His, G S T, and Myc, and it may also be fused with other polypeptides suitable for promoting the antigen detection.

In one or more implementation schemes, the present disclosure includes a nucleic acid sequence containing the antibody or the fragment thereof for encoding the anti-Plasmodium falciparum HRP-II, In this article, the nucleic acid sequence includes a conservatively substituted variant thereof (for example, substitution of a degenerate codon) and a complementary sequence. Terms “nucleic acid” and “polynucleotide” are synonymous and include a gene, a cDNA molecule, an mRNA molecule and their fragments such as an oligonucleotide.

In one or more implementation schemes, the present disclosure includes an expression vector containing the nucleic acid sequence for encoding the anti-Plasmodium falciparum HRP-II antibody or the fragment thereof, herein the nucleic acid sequence is operably linked to at least one regulatory sequence. “Operably linked” means that an encoding sequence is connected to a regulatory sequence in a mode that the expression of the coding sequence is allowed. The regulatory sequence is selected to be used to direct the expression of the target protein in a suitable host cell, including a promoter, an enhancer and other expression regulatory elements.

In this article, the vector includes a molecule or an agent that contains the nucleic acid or the fragment thereof of the present disclosure, may carry genetic information and may deliver the genetic information into a cell. A typical vector includes a plasmid, a virus, a bacteriophage, a cosmid, and a mini-chromosome. The vector may be a cloning vector (namely, a vector used to transfer the genetic information into the cell, the cell may be propagated and the cell with or without the genetic information may be selected) or an expression vector (namely, a vector that contains a necessary genetic element so that the genetic information of the vector is allowed to be expressed in the cell). Therefore, the cloning vector may include a selection marker, and an origin of replication which is matched with the cell type specified by the cloning vector, and the expression vector may include the regulatory element necessary for affecting the expression in the specified target cell.

The nucleic acid or the fragment thereof of the present disclosure may be inserted into a suitable vector to form the cloning vector or the expression vector carrying the nucleic acid fragment of the present disclosure. In one or more implementation schemes, the vector may include a plasmid, a bacteriophage, a cosmid, a minichromosome, or a virus, as well as a naked DNA that is only transiently expressed in the specific cells. In one or more implementation schemes, the vector contains a sequence carrying a target gene and a screening label (such as Dhfr, a GS label, or resistance genes neo, and hygro). In one or more implementation schemes, the vector of the present disclosure includes 1) a monocistronic vector, for example, the heavy chain and light chain of the antibody use the different vectors to co-transfect the host cell; the heavy chain and light chain genes may also be serially connected to the same vector, and respective promoter and terminator are respectively used for transcription and translation; 2) a bicistronic vector, for example, a ribosome internal entry site is constructed in the vector, and the light chain and heavy chain genes are serially connected at both ends thereof to achieve simultaneous transcription and translation of the light and heavy chains; 3) a trans-complementary vector, for example, two vectors for expressing the light and heavy chains respectively contain different sequences for encoding screening label genes, only if the light and heavy chains are transfected at the same time, the screening label may be correctly expressed, thereby the screening efficiency is improved. Any methods may be used to introduce mutations into the coding sequence to generate the variant of the present disclosure, and these mutations may include deletion or insertion or substitution and the like.

The expression vector of the present disclosure is used to transform the host cell. Such a transformed cell is also a part of the present disclosure, and may be used for proliferating the nucleic acid fragment and the vector of the present disclosure, or used for recombinantly preparing a culture cell or a cell line of the polypeptide of the present disclosure. The host cell of the present disclosure includes any engineered cells suitable for preparing the recombinant antibody. In one or more implementation schemes, the host cell may include microorganisms such as bacteria (such as Escherichia coli, and Bacillus), multicellular organisms such as a yeast cell, an insect cell, a plant cell or a mammalian cell, preferably cells derived from humans. In one or more implementation schemes, the host cell may be used to prepare the antibody described in this article, such as a single-chain antibody or an antibody fragment, or the separated polypeptide, the fusion protein and the like. In one or more implementation schemes, the host cell of the present disclosure includes the mammalian cell suitable for preparing the recombinant antibody, such as a CHO, a myeloma cell, a hybridoma cell, PerC.6, HEK293, NS0, and sp2/0. In one or more implementation schemes, the host cell preferably includes a cell with good growth, high serum-free suspension culture density, strong heterologous expression ability, and correct post-translational modification ability. In one or more implementation schemes, the present disclosure provides a preparation method for an anti-Plasmodium falciparum HRP-II antibody, an antigen-binding fragment, a variant and a functional derivative. In one or more implementation schemes, the anti-Plasmodium falciparum HRP-II monoclonal antibody is expressed by a recombinant technology. In one or more implementation schemes, the host cell is transfected with a nucleic acid vector for encoding the anti-Plasmodium falciparum HRP-II antibody, and the host cell is cultured under suitable conditions to express the anti-Plasmodium falciparum HRP-II antibody. The host cell may also be transfected with one or more expression vectors, the expression vector may contain a DNA for encoding at least part of the anti-Plasmodium falciparum HRP-II antibody alone or conjunctively. In one or more implementation schemes, while the antibody of the present disclosure is recombinantly prepared, the expression product may be exported to a culture medium or carried on the surface of the transformed cell. A conventional technology for purification of the protein and peptide may be used to separate the anti-Plasmodium falciparum HRP-II antibody from the culture medium or a cell lysate. The technology includes ammonium sulfate precipitation, chromatography (such as ion exchange, gel filtration, affinity and chromatography) and/or electrophoresis. In one or more implementation schemes, an immunogen used to prepare the antibody may contain an intact Plasmodium falciparum HRP-II, or a fragment or derivative thereof. The preferred immunogen contains all or part of the Plasmodium falciparum HRP-II.

In one or more implementation schemes, the antibody described in this article, such as the anti-Plasmodium falciparum HRP-II antibody, may be used to detect the presence of one or more target molecules, such as the HRP-II, in a biological sample. A term “detection” while being used in this article includes quantitative or qualitative detection. In one or more implementation schemes, the biological sample includes a cell or a tissue.

In one or more implementation schemes, a method for diagnosis or detection is provided, the method includes contacting the biological sample with the antibody, such as the anti-Plasmodium falciparum HRP-II antibody, described in this article under a condition that the antibody is allowed to bind to a target, and detecting whether a complex is formed between the antibody and the target. In one or more implementation schemes, the method may be an in vitro or in vivo method. The present disclosure also relates to a method for diagnosing malaria in a subject who may be infected with the Plasmodium falciparum, and it includes contacting a biological sample from the subject with the antibody of the present disclosure, and the contact is performed under a condition that the antibody is enabled to form an antigen/antibody complex with the target which may exist in the biological sample, and the possibly formed antigen/antibody complex is detected. In this method, in vitro diagnosis may be performed by ELISA analysis. In one or more implementation schemes, the method of the present disclosure may further include contacting the biological sample with one or more antibodies that diagnose other Plasmodium antigens, the other antigens may be, for example, LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP.

In one or more implementation schemes, a labeled anti-Plasmodium falciparum HRP-II antibody is provided. In one or more implementation schemes, the label includes, but is not limited to, a fluorescent label, a chromophore label, an electron-dense label, a chemiluminescent label, and a radioactive label, as well as an indirect label such as an enzyme or a ligand, for example, indirect detection is performed by an enzymatic reaction or a molecular interaction. In one or more implementation schemes, an exemplary label includes, but is not limited to, a radioisotope, a fluorophore, rhodamine and a derivative thereof, a luciferase, a luciferin, a horseradish peroxidase (HRP), an alkaline phosphatase, a P-galactosidase, a glucoamylase, a lysozyme, a carbohydrate oxidase, for example, a glucose oxidase, a galactose oxidase, and a glucose-6-phosphate dehydrogenase, biotin/avidin, a spin label, a phage label and the like.

In one or more implementation schemes, the anti-Plasmodium falciparum HRP-II antibody or the fragment thereof of the present disclosure may be used as the antigen in immunoassay and corresponding kit for HRP-II antibody detection. In one or more implementation schemes, the immunoassay of the present disclosure includes an ELISA, and an indirect immunofluorescence assay IFA, and further includes a radioimmunoassay RIA, and other non-enzyme-linked antibody binding assays or methods.

In one or more implementation schemes, such as in an ELISA double antigen antibody sandwich experimental scheme, the HRP-II antibody may be coated on a micro-reaction plate, the HRP-II antigen in a sample may be captured, and then the labeled antibody may be used to recombine with the antigen combined on the reaction plate, and a result is read after color development. In one or more implementation schemes, the HRP-II antibody of the present disclosure may be used to coat the micro-reaction plate or used as a labeled second antibody. In one or more implementation schemes, the anti-Plasmodium falciparum HRP-II antibody or the fragment thereof is immobilized on a surface, such as a solid-phase support, such as plastic, a membrane such as a nitrocellulose membrane, a glass or metal support. In one or more implementation schemes, the solid-phase support may be made into a strip shape or a card shape. In one or more implementation schemes, a sample from the subject contacts with the solid-phase support, and then contacts with an antibody indicator with a detectable label, such as a rhodamine label, for the color development. In one or more implementation schemes, blocking agents such as bovine serum albumin, milk powder solution, gelatin, PVP, and Superblock may be used to block non-specific sites, thereby the background caused by non-specific binding is reduced. In one or more implementation schemes, a diluent may be used, for example, BSA and phosphate buffered saline (PBS)/Tween are used to dilute antiserum, it is helpful to reduce the non-specific background.

In one or more implementation schemes, this article relates to a method for determining the presence or quantification of the HRP-II antigen. In one or more implementation schemes, the method includes contacting a biological sample that may contain the HRP-II antigen with the anti-Plasmodium falciparum HRP-II antibody of the present disclosure, and quantitatively or qualitatively determining the binding between at least one anti-Plasmodium falciparum HRP-II antibody and the antigen in the sample. The method may take any forms, and may be, for example, a non-competitive or competitive ELISA, a RIA or magnetic immunoassay, an agglutination assay, and a surface plasmon resonance-based assay such as a Biacore assay.

In this article, the biological sample may refer to a sample of a biological tissue, a cell, or fluid such as body fluid in a healthy and/or pathological state. In one or more implementation schemes, the biological sample is from a subject suspected to be infected with the malaria. In one or more implementation schemes, the biological sample may be a body fluid sample, such as a sample of cerebrospinal fluid, urine, saliva, and blood.

In one or more implementation schemes, this article provides a test or diagnostic device or a related kit, it may include:

(i) an anti-Plasmodium falciparum HRP-II antibody that may bind to an HRP-II antigen; and

(II) an indicator for indication while the antigen binds to the anti-Plasmodium falciparum HRP-II antibody.

In one or more implementation schemes, the kit may also include an antibody for other Plasmodium antigens in addition to the anti-Plasmodium falciparum HRP-II antibody of the present disclosure, for example, it is used for identifying different Plasmodium infections. In one or more implementation schemes, other antigens include, for example, Plasmodium falciparum, Plasmodium vivax, Plasmodium malariae and/or Plasmodium ovale specific antigens or shared antigens, such as an aldolase, such as a lactate dehydrogenase PLDH, such as a Plasmodium lactate dehydrogenase PLDH or a Plasmodium species-specific lactate dehydrogenase PLDH, such as a Plasmodium glutamate dehydrogenase PGDH, LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP.

In one or more implementation schemes, the kit may include a test strip or a test card, the liquid sample from the subject is placed on the test strip, or an ELISA assay plate, the ELISA assay plate has a hole in which a liquid sample from a single subject may be placed. In one or more implementation schemes, the kit may include a test device configured for use in a flow cytometer, a bioanalyzer, and a biosensor.

In one or more implementation schemes, the kit is a Rapid Diagnostic Test (RDT) kit, such as a kit for thin-film immunochromatography, such as a kit for thin-film immunochromatography using a colloidal gold method, such as a kit containing a solid-phase support such as a membrane support, the antibody which binds to the Plasmodium antigen or the antigen-binding fragment thereof, the antibody conjugate, or the fusion protein and an optional control antibody are immobilized to the solid-phase support. In one or more implementation schemes, the kit, such as the Rapid Diagnostic Test (RDT) kit, may include a test strip or a test card, and the test strip or test card may include a sample pad, a label pad which is adjacent to the sample pad and contains the Plasmodium antibody (it may include one or more antibodies, such as the anti-Plasmodium falciparum HRP-II antibody as described in this article and the antibody in allusion to other Plasmodium antigens, the antibody may have a detectable label such as a colloidal gold), and a membrane adjacent to the label pad, such as a cellulose membrane and an adjacent water absorbent pad. In one or more implementation schemes, a test line and a quality control line that are separated from each other may be installed on the membrane, and the test line contains a Plasmodium-specific antibody that binds to different epitopes with the labeled antibody, and the quality control line may contain an antibody that may specifically bind to the labeled antibody. In one or more implementation schemes, the test strip or test card may include a support, a sample pad, a label pad, a membrane such as a nitrocellulose membrane, an absorbent pad, one or more test lines, and a quality control line. The sample pad, the label pad, the nitrocellulose membrane, and the absorbent pad are sequentially arranged from one end of the support to the other end, the sample pad and the label pad are partially overlapped, the label pad and the nitrocellulose membrane are partially overlapped, the nitrocellulose membrane and the absorbent pad are partially overlapped, the one or more detection lines and the quality control line are respectively arranged on the nitrocellulose membrane at intervals, and the label pad may be coated with a detectable label such as the Plasmodium antibody labeled by the colloidal gold, the one or more detection lines may contain the Plasmodium specific antibody that binds to the different epitopes with the labeled antibody, and the quality control line may contain the antibody that specifically binds to the labeled antibody.

In one or more implementation schemes, the present disclosure provides a kit, and it contains a material which may be used for diagnosing Plasmodium infection. In one or more implementation schemes, the kit includes a container and a label or a packaging specification on or with the container. In one or more implementation schemes, the suitable container includes, for example, a bottle, a vial, and a syringe. The container may be made of various materials such as glass or plastic. A composition is loaded in the container, and the composition is alone or combined with another composition which is effectively used for diagnosing the Plasmodium infection. At least one active agent in the composition is the anti-Plasmodium falciparum HRP-II antibody of this article. In addition, the kit may include: (a) a first container containing the composition, herein the composition contains the anti-Plasmodium falciparum HRP-II antibody of this article; and (b) a second container containing the composition, herein the composition includes a second antibody and/or other related reagents. In one or more implementation schemes, the kit may include an antibody in allusion to other malaria antigens in addition to the anti-Plasmodium falciparum HRP-II antibody of the present disclosure, for example, it is used to identify the different Plasmodium infections. The kit of the present disclosure may also include a packaging specification, and the packaging specification indicates that the composition may be used to diagnose the disease or infection. The kit may also include a second or third container, the second or third container contains a buffer, such as water for injection, phosphate buffered saline, glucose solution, and may also include other materials, such as other buffers, a dilutent, a filter, a needle and a syringe.

In one or more implementation schemes, this article provides a kit containing the anti-Plasmodium falciparum HRP-II antibody, such as a microtiter plate (it may be coated with the anti-Plasmodium falciparum HRP-II antibody). In one or more implementation schemes, the microtiter plate may include a titration well such as a polystyrene microtitre well, and these sampling wells are coated with the anti-Plasmodium falciparum HRP-II antibody, or a complete cell of the Plasmodium infection, or a cell lysate.

In one or more implementation schemes, this article provides a kit for determining, for example, a subject infected with Plasmodium, the kit contains at least one anti-Plasmodium falciparum HRP-II antibody of the present disclosure, a related buffer, a reagent required for reacting a liquid sample with the anti-Plasmodium falciparum HRP-II antibody, and a reagent for determining whether there is a positive or negative binding reaction between the antibody and the anti-Plasmodium falciparum HRP-II antibody. Such a kit may include a container for separating and/or storing the liquid sample, a container and reagent for contacting the anti-Plasmodium falciparum HRP-II antibody with the sample, and a reagent for determining the binding between the anti-Plasmodium falciparum HRP-II antibody and components in the sample. In order to determine the presence of the antibody, the kit may, for example, use the antibody or antigen with a second label (double antigen sandwich method), herein the label may be any suitable labels, such as a fluorescent or radioactive label, an enzyme label or a binding label, such as a biotin label for binding to streptavidin. In the kit, the anti-Plasmodium falciparum HRP-II antibody may be combined with a solid-phase support, or the kit may at least include the solid-phase support suitable for binding to the anti-Plasmodium falciparum HRP-II antibody.

In one or more implementation schemes, the HRP-II antibody of the kit may be a form of liquid solution, a form attached to the solid-phase support, or dry powder. While the HRP-II antibody reagent is liquid solution, the liquid solution may be aqueous solution. While the reagent is the form attached to the solid-phase support, the preferred solid-phase support may be a chromatographic medium such as a film, a test strip, a plastic bead or plate, or a microscope slide. While the reagent is dry powder, the powder may be reconstituted by adding a suitable solvent. In one or more implementation schemes, the kit may further include a container containing the suitable solvent.

In one or more implementation schemes, the kit includes a container, it includes a quantitative second antibody, such as a second antibody coupled with the alkaline phosphatase, and a second container, herein it includes a certain amount of a buffer. In other implementation schemes, the kit may further include a third container, it includes a suitable substrate, such as PNPP for the alkaline phosphatase, or a substrate for the peroxidase. A fourth container may include an appropriate “stop” buffer.

### Embodiment

1. Expression Plasmid Construction

In the embodiment, a restriction endonuclease and a Prime Star DNA polymerase are purchased from Takara Company. An MagExtractor-RNA extraction kit is purchased from TOYOBO Company. A BD SMART™ RACE cDNA Amplification Kit is purchased from Takara Company. A pMD-18T vector is purchased from Takara Company. A plasmid extraction kit is purchased from Tian'gen Company. Primer synthesis and gene sequencing are completed by Invitrogen Company. A hybridoma cell line that secretes an Anti-HRP-II 3R5 monoclonal antibody is an existing hybridoma cell line. It is obtained by a spleen cell of a mouse immunized with an MA-HRPII Ag protein (MyBioSource, MBS319418) fused with a SP 210 cell through screening, the cells are quickly recovered from liquid nitrogen and added to a complete medium (20% fetal bovine serum+80% RPMI 1640) for use in culture.

1.1. Primer

Amplification Heavy Chain and Light Chain 5′RACE Primers:

SMARTER II A Oligonucleotide:
(SEQ ID NO. 7)
5′>AAGCAGTGGTATCAACGCAGAGTACXXXXX<3′;
5′-RACE CDS Primer (5′-CDS):
(SEQ ID NO. 8)
5′>(T) 25VN<3′(N = A, C, G, or T; V = A, G, or
C);
Universal Primer A Mix (UPM):
(SEQ ID NO. 9)
5′>CTAATACGACTCACTATAGGGCAAGCAGTGGTATCAACGCAGAG
T<3′
Nested Universal Primer A(NUP):
(SEQ ID NO. 10)
5′>AAGCAGTGGTATCAACGCAGAGT<3′
mlgG CKR:
(SEQ ID NO. 11)
5′>CGCCTAACACTCATTCCTGTTGAAGC<3′.
mlgG CHR:
(SEQ ID NO. 12)
5′>CCGCTCATTTACCCGGAGACCG<3′.

1.2. Antibody Variable Region Gene Cloning and Sequencing

A RNA is extracted from a hybridoma cell line that secretes the Anti-HRP-II 3R5 monoclonal antibody, and the SMARTER™ RACE cDNA Amplification Kit and SMARTER II A Oligonucleotide and 5′-CDS primers in the kit are used for first chain cDNA synthesis according to a scheme recommended by a manufacturer, and a first chain cDNA product obtained is used as a PCR amplification template. A Light Chain gene is amplified with Universal Primer A Mix (UPM), Nested Universal Primer A (NUP) and mlgG CKR primers, and a Heavy Chain gene is amplified with Universal Primer A Mix (UPM), Nested Universal Primer A (NUP) and mIgG CHR primers. Herein about 0.8 KB of a target band is amplified by a primer pair of Light Chain, and about 1.4 KB of a target band is amplified by a primer pair of Heavy Chain. It is purified and recovered by agarose gel electrophoresis, a product is subjected to an A-adding reaction with a rTaq DNA polymerase and inserted into a pMD-18T vector, and transformed into a DH5a competent cell. After a bacterial colony grows, and cloned. 4 clones of each the Heavy Chain and Light Chain genes are respectively taken and sent to Invitrogen Company for sequencing.

1.3. Sequence Analysis of Variable Region Genes of Anti-HRP-II 3R5 Antibody

The gene sequence obtained by the above sequencing is put in an IMGT antibody database for analysis, and VNTI11.5 software is used to analyze and determine that the genes amplified by the heavy chain and light chain primer pairs are all correct, herein in a gene fragment amplified by the Light Chain, a VL gene sequence is 405 bp, and belongs to a VkII gene family, there is 66 bp of a leader peptide sequence in front of it; and in a gene fragment amplified by the Heavy Chain primer pair, a VH gene sequence is 399 bp, and belongs to a VH1 gene family, there is 57 bp of a leader peptide sequence in front of it. The antibody heavy chain and light chain sequences are shown in SEQ ID NOs: 35-38.

1.4. Construction of Recombinant Antibody Expression Plasmid

pcDNA™ 3.4 TOPO® vector is a constructed recombinant antibody eukaryotic expression vector, purchased from Thermo Scientific. This polyclonal restriction sites such as HindIII, BamHI, and EcoRI have been introduced into the expression vector, which named as a pcDNA3.4A expression vector, and then referred to as a 3.4A expression vector; according to the above antibody variable region gene sequencing result in pMD-18T, VL and VH gene-specific primers of the Anti-HRP-II 3R5 are designed, and respectively provided with HindIII, EcoRI restriction sites and a protective base at both ends, the primers are as follows:

HRP-II3R5-HF:
(SEQ ID NO. 13)
5′>CCCAAGCTTGCCACCATGTACTTGGGACTGAACTGTGTATTC<3′;
HRP-II3R5-HR:
(SEQ ID NO. 14)
5′>GGGGAATTCTCATTACTTTCCGGGAGATCTGGAGATGGTTTGCTT
G<3′;
HRP-II3R5-LF:
(SEQ ID NO. 15)
5′>CCCAAGCTTGCCACCATGGACATCAGGGCTCCTGCTCAGTTTCTTGG
C<3′;
HRP-II3R5-LR:
(SEQ ID NO. 16)
5′>GGGGAATTCTCATTAACATTCATTTCTGTTAAAAGATTTGACAAT
G<3′;

A Light Chain gene fragment and a Heavy Chain gene fragment are amplified from the above cloned genes by a PCR amplification method. The Heavy Chain and Light Chain gene fragments are double-digested with HindIII/EcoRI, respectively, and a 3.4A vector is double-digested with the HindIII/EcoRI. After the fragment and vector are purified and recovered, the Heavy Chain gene and Light Chain gene are connected to the 3.4A expression vector respectively, the recombinant expression plasmids of Heavy Chain and Light Chain are respectively obtained.

2. Antibody Preparation

2.1. Transient-Transfected CHO Cells of Recombinant Antibody Expression Plasmid and Antibody Activity Identification of Expression Supernatant

2.1.1. Transient-Transfected CHO Cells of Recombinant Plasmid

The 3.4A recombinant expression plasmid containing antibody light and heavy chain DNAs constructed in the above Embodiment 1 is diluted to 400 μg/ml with ultrapure water, CHO cells are adjusted to 1.7×10⁷ cells/ml in a centrifuge tube, 100 μl of the plasmid is mixed with 700 μl of the cells, and transferred to an electroporation cup for electroporation, and transferred to 10 ml of a CD CHO AGT (Thermo Scientific) medium, it is cultured in a shaker at 37° C. (8% CO₂, vibration amplitude 115-200 rpm); a sample is taken everyday to detect a cell viability, while the cell viability is less than 50%, the cells are centrifuged and supernatant is cultured.

2.1.2. Antibody Activity Identification of Expression Supernatant

The HRP-II protein is diluted to 1 ng/ml with 50 mM carbonate buffer, 100 uL per well, and it is coated overnight at 4° C.; on the next day, it is washed twice with PBST washing solution and patted dry; blocking solution (20% BSA+80% PBS) is added, 120 uL per well, and it is incubated at 37° C. for 1 h, and patted dry; the cell supernatant diluted by multiples is added, 100 uL/well, and it is incubated at 37° C. for 30 min (partial supernatant 1 h); it is washed for 5 times with the PBST washing solution, and patted dry; a goat anti-mouse IgG-HRP (Abcam produced by the company itself, ab97019) is added, 100 uL per well, 37° C., and 30 min; it is washed for 5 times with the PBST washing solution, and patted dry; carbamide peroxide (50 uL/well), and tetramethyl benzidine (50 uL/well) are added, and the color development is performed for 10 min; dilute hydrochloric acid is added to stop a reaction, 50 uL/well; and an OD value is read at 450 nm (refer to 630 nm) on a microplate reader.

Anti-HRP-H3R5 CHO transient-transfected cell
supernatant antibody activity identification

| | Dilution ratio | | | | | | | | |
|---|---|---|---|---|---|---|---|---|---|
| | | | | | | | | | Blank |
| | 1/1 | 1/5 | 1/25 | 1/125 | 1/625 | 1/3125 | 1/15625 | 1/78125 | well |
| CHO | 2.151 | 1.975 | 1.613 | 1.15 | 0.732 | 0.395 | 0.233 | 0.151 | 0.122 |
| transient-transfected | | | | | | | | | |
| cell supernatant | | | | | | | | | |
| Antibody prepared | 2.203 | 2.132 | 1.978 | 1.653 | 1.239 | 0.865 | 0.503 | 0.321 | 0.102 |
| by hybridoma | | | | | | | | | |

Note:
a reference substance is diluted by 2 times successively from 100 ng/ml, and there is no reference substance in the blank well.

It may be seen from results of the above table that the antibody generated after transient transfection of the constructed 3.4A recombinant expression plasmid has good activity on the HRP-II protein.

2.2. Antibody Purification and Affinity Analysis, Activity Identification

2.2.1. Expression Supernatant Purified by Protein A Affinity Chromatography Column

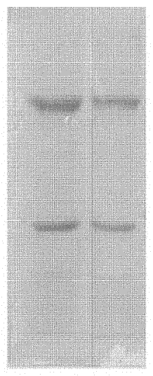

Supernatant of fermentation solution is taken and filtered with 0.22 pm of a membrane. The supernatant passes through a Mab Select SuRe LX (GE Healthcare) affinity packing column at a certain flow rate and hanged on the column, and then 20 mM NaAc (pH3.4) solution is used for elution, and a certain amount of 1 M Tris solution is added to a sample collection tube for pre-neutralization. The eluted sample is dialyzed in PBS (pH 7.4) solution for three times and then a purified antibody is obtained. 5 μg of the purified antibody is taken for performing reducibility SDS-PAGE. Results are shown in FIG. 1, and lanes 1 and 2 are all 5 ug of a loading amount.

2.2.2 Affinity Analysis of Purified Antibody

Affinity Analysis

Data is made with an enzyme immunoassay indirect method in the same way of the activity identification, and coating is made into four gradients of 1 ug/ml, 0.5 ug/ml, 0.25 ug/ml, and 0.125 ug/ml; the antibody is diluted by 2 times of the gradient from 1000 ng/ml to 0.97656 ng/ml and a sample is loaded. The OD values corresponding to different antibody concentrations with different coating concentrations are obtained. Under the same coating concentration, the antibody concentrations are used as an abscissa, the OD values are used as an ordinate, and a logarithmic plot is made. According to a fitting equation, the antibody concentration at 50% of the maximum OD value is calculated; it is assigned into a formula: K=(n−1)/(2*(n*Ab′−Ab)), a reciprocal of an affinity constant is calculated, herein Ab and Ab′ respectively represent the antibody concentrations at 50% of the maximum OD value in the corresponding coating concentrations (Ag, Ag′), n=Ag/Ag′; every two coating concentrations may be combined to calculate a K value, and finally six K values may be obtained. An average value thereof is taken and a reciprocal thereof is calculated to get the affinity constant KD.

Affinity Analysis Data of Purified Anti-HRP-II 3R5 Monoclonal Antibody

| Sample name | KD |
|---|---|
| Anti-HRP-II3R5 | 6.334E−10 |

2.2.3. Activity Identification of Purified Antibody

The MA-HRPII Ag recombinant MA protein (MyBioSource, MBS319418) (self-produced 150520-1) is diluted with 50 mM carbonate buffer coating solution to 1 ug/mi for microplate coating, 100 uL per well, overnight at 4° C.; on the next day, it is washed twice with PBST, and patted dry; blocking solution (20% BSA+80% PBS) is added, 120 uL per well, 37° C., and 1 h, and patted dry; the diluted Anti-HRP-II 3R5 monoclonal antibody is added, and 5-fold dilution is performed from 1000 ng/ml, a sample is loaded, 100 uL/well, 37° C., and 30 min (partial supernatant 1 h); it is washed for 5 times with the PBST washing solution, and patted dry; goat anti-mouse IgG-HRP is added, 100 uL per well, 37° C., and 30 min; it is washed for 5 times with the PBST washing solution, and patted dry; the carbamide peroxide (50 uL/well) and the tetramethyl benzidine (50 uL/well) are added, 10 min; the dilute hydrochloric acid is added to stop a reaction, 50 uL/well; and the OD value is read at 450 nm (refer to 630 nm) on the microplate reader.

Activity Identification of Purified Anti-HRP-II 3R5 Monoclonal Antibody

| | Sample concentration ng/ml | | | | | | |
|---|---|---|---|---|---|---|---|
| | 1000 | 200 | 40 | 8 | 1.6 | 0.32 | 0 |
| Anti-HRP-II3R5 | 2.336 | 1.971 | 0.402 | 0.122 | 0.053 | 0.058 | 0.087 |

An anti-Plasmodium falciparum HRP-II monoclonal antibody sequence is provided, and the Anti-HRP-II monoclonal antibody with activity and higher affinity is obtained through the recombinant antibody technology, it advantageously has the following advantages:

1) Compared with the hybridoma technology, the CHO cell line is more stable, and it is not easy to lose an antibody secretion ability during processes of passage, cryopreservation and recovery, and it is easier to store a precious cell line.

2) The expression vector of the anti-HRP-II recombinant antibody is successfully constructed, it may be expressed in the CHO cells and has the biological activity. Compared with the hybridoma technology, a recombinant antibody expression yield of the CHO cell line may reach about 2 g/L, and a cost is lower in mass production; and in addition, the cell line is more stable, the production stability is good, a batch difference is smaller, purification difficulty is smaller, and the cost is less.

3) The recombinant antibody technology is used to construct the expression vector that may specifically recognize an epitope of a Plasmodium falciparum HRP-II protein antigen, and a specific monoclonal antibody is generated, the occurrence of a non-specific reaction is reduced, and the specificity of detecting the Plasmodium falciparum is improved.

3. Mutation of light chain CDR and heavy chain CDR of antibody (having the light chain and the heavy chain shown in SEQ ID NO: 20 and 18, as WT) obtained in above Embodiments.

After Analysis, the Complementarity Determining Region (WT) of the Heavy Chain:

### CDR-VH1 is G-X1(S)-T-X2(L)-T-X3(E)-Y-Y-M-N;

### CDR-VH2 is D-X1(L)-N-P-l-X2(N)-G-G-T-X3(P)—Y-X4(Q)-Q-K-F; and

### CDR-VH3 is T-X1(R)-G-A-E-X2(E)-Y;

the complementarity determining region of the light chain:

### CDR-VL1 is S-Q-S-X1(II)-Y-S-X2(N)-G-K-I-Y-X3(I)-N;

### CDR-VL2 is Q-X1(L)-S-K-X2(I)-X3(D)-P; and

### CDR-VL3 is L-Q-X1 (G)-T-Y-X2(S)-P-X3(N)-T,

herein, X1, X2, X3, and X4 are all mutation sites.

### Mutation Site Design

| | CDR-VH1 | CDR-VH2 | CDR-VH3 | CDR-VL1 | CDR-VL2 | CDR-VL3 |
|---|---|---|---|---|---|---|
| Site | X3 | X3 | X2 | X3 | X2 | X3 |
| WT | E | P | E | I | I | N |
| Mutation 1 | D | A | D | L | L | Q |
| Mutation 2 | E | P | E | L | I | N |
| Mutation 3 | Q | V | M | G | F | M |
| Mutation 4 | V | Q | K | A | W | R |
| Mutation 5 | R | G | W | R | K | K |

After mutation, the antibody activity is detected. The MA-HRPII Ag protein (Feipeng Biotechnology Company, 150520-1) is diluted with the coating solution to 1 ug/ml for microplate coating, 100 uL per well, and overnight at 4° C.; on the next day, it is washed twice with the washing solution, and patted dry; the blocking solution (20% BSA+80% PBS) is added, 120 uL per well, 37° C., and 1 h, and patted dry; the diluted Anti-HRP-II 3R5 monoclonal antibody is added, 100 uL/well, 37° C., and 30 min (partial supernatant 1 h); it is washed for 5 times with the washing solution, and patted dry; the goat anti-mouse IgG-HRP is added, 100 uL per well, 37° C., and 30 min; it is washed for 5 times with the washing solution, and patted dry; urea peroxide (50 uL/well) and tetramethyl benzidine (50 uL/well) are added and the color development is performed for 10 min; the dilute hydrochloric acid is added to stop a reaction, 50 uL/well; and the OD value is read at 450 nm (refer to 630 nm) on the microplate reader, Some results are as follows:

### Antibody Activity Analysis Data

| Antibody concentration (ng/ml) | WT | Mutation 1 | Mutation 2 | Mutation 3 | Mutation 4 | Mutation 5 |
|---|---|---|---|---|---|---|
| 1000 | 2.336 | 2.516 | 2.226 | 0.814 | 0.518 | — |
| 200 | 1.971 | 2.405 | 2.052 | 0.635 | 0.065 | — |
| 40 | 0.402 | 0.695 | 0.551 | 0.032 | — | — |
| 8 | 0.122 | 0.288 | 0.189 | — | — | — |
| 1.6 | 0.053 | 0.160 | 0.101 | — | — | — |
| 0.32 | 0.058 | 0.089 | 0.077 | — | — | — |
| 0 | 0.087 | 0.084 | 0.081 | — | — | — |

“—” represents no activity.

It may be seen from the above table that Mutation 1 and Mutation 2 have the better activity, especially Mutation 1 has the best activity. Furthermore, Mutation 1 is used as a framework sequence to screen a mutation site with a better titer (it is guaranteed that the antibody activity obtained by screening is similar to that of Mutation 1, and the antibody activity is ±10%). Some results are as follows.

List of Mutation Sites with Better Titer

| | CDR-VH1 | CDR-VH2 | CDR-VH3 | CDR-VL1 | CDR-VL2 | CDR-VL3 |
|---|---|---|---|---|---|---|
| Site | X1/X2 | X1/X2/X4 | X1 | X1/X2 | X1/X3 | X1/X2 |
| Mutation 1-0 | Y/L | L/Q/N | K | LL/Q | I/D | A/S |
| Mutation 1-1 | Y/I | I/Q/N | R | II/Q | I/E | G/Y |
| Mutation 1-2 | S/L | V/Q/Q | K | LL/N | L/D | A/T |
| Mutation 1-3 | Y/l | L/N/Q | R | II/N | L/E | G/S |
| Mutation 1-4 | Y/L | I/N/Q | K | IL/N | V/D | A/Y |
| Mutation 1-5 | S/L | V/N/N | R | IL/Q | V/E | G/T |
| Mutation 1-6 | Y/I | L/Q/N | K | LI/N | I/D | A/S |
| Mutation 1-7 | Y/I | I/N/Q | R | LI/N | I/E | A/Y |
| Mutation 1-8 | Y/I | V/Q/N | K | LL/Q | L/D | A/T |
| Mutation 1-9 | Y/L | L/Q/N | R | II/Q | L/E | G/S |
| Mutation 1-10 | Y/I | I/N/N | R | LL/N | V/D | G/Y |
| Mutation 1-11 | Y/I | V/N/N | K | II/N | V/E | A/T |
| Mutation 1-12 | Y/I | L/N/N | R | IL/N | I/D | G/S |
| Mutation 1-13 | S/L | I/Q/N | K | IL/Q | I/E | G/Y |
| Mutation 1-14 | Y/L | V/N/N | K | LI/N | L/D | A/T |
| Mutation 1-15 | Y/I | L/Q/N | R | LI/N | L/E | G/T |
| Mutation 1-16 | Y/I | I/Q/N | R | LL/Q | V/D | G/S |
| Mutation 1-17 | Y/I | V/N/N | K | LL/Q | V/E | G/Y |
| Mutation 1-18 | Y/L | L/Q/N | K | LL/Q | I/D | G/T |
| Mutation 1-19 | T/L | I/Q/N | R | II/Q | I/E | A/S |
| Mutation 1-20 | S/I | V/Q/N | K | II/Q | L/D | G/Y |
| Mutation 1-21 | T/L | L/Q/N | R | LL/Q | L/E | G/T |
| Mutation 1-22 | Y/I | I/Q/Q | K | II/N | V/D | A/S |
| Mutation 1-23 | T/L | V/Q/N | R | II/N | V/E | G/Y |
| Mutation 1-24 | T/L | L/Q/Q | K | II/N | I/D | A/S |
| Mutation 1-25 | Y/I | I/Q/N | R | LL/Q | I/E | G/T |
| Mutation 1-26 | T/L | V/Q/N | R | LL/Q | L/D | G/Y |
| Mutation 1-27 | T/L | L/N/Q | K | II/N | L/E | G/S |
| Mutation 1-28 | Y/I | I/Q/Q | K | II/N | V/D | A/T |
| Mutation 1-29 | Y/I | V/Q/N | R | II/Q | V/E | G/Y |
| Mutation 1-30 | S/L | L/Q/N | K | II/N | I/D | A/S |
| Mutation 1-31 | T/L | I/N/Q | K | LL/N | I/E | G/T |
| Mutation 1-32 | Y/I | V/Q/Q | R | LL/N | L/D | A/Y |
| Mutation 1-33 | T/L | L/Q/N | K | LL/N | L/E | G/S |
| Mutation 1-34 | T/L | I/N/Q | R | LL/N | V/D | G/T |
| Mutation 1-35 | Y/I | V/Q/Q | R | LL/N | V/E | A/Y |
| Mutation 1-36 | T/L | L/Q/N | R | LL/N | I/D | G/S |
| Mutation 1-37 | Y/I | I/N/Q | K | LL/N | I/E | A/Y |
| Mutation 1-38 | T/L | V/Q/Q | R | LL/N | L/D | G/S |
| Mutation 1-39 | T/L | L/N/Q | K | LL/N | L/E | A/T |
| Mutation 1-40 | T/L | I/N/Q | R | IL/N | L/D | G/T |
| Mutation 1-41 | Y/L | V/N/Q | K | IL/N | V/E | A/Y |
| Mutation 1-42 | Y/I | L/N/Q | R | IL/N | I/D | G/S |
| Mutation 1-43 | T/L | I/N/Q | K | IL/N | I/E | G/S |
| Mutation 1-44 | Y/I | V/N/Q | R | IL/N | L/D | A/S |
| Mutation 1-45 | Y/I | L/N/N | K | IL/Q | L/E | G/Y |
| Mutation 1-46 | Y/I | I/N/N | R | IL/Q | V/D | G/T |
| Mutation 1-47 | S/I | V/N/N | K | IL/Q | V/E | A/S |
| Mutation 1-48 | Y/I | L/Q/N | R | LL/Q | I/D | G/Y |
| Mutation 1-49 | Y/L | I/Q/N | K | LL/Q | I/E | A/T |
| Mutation 1-50 | Y/I | V/Q/Q | R | LL/N | L/E | A/Y |
| Mutation 1-51 | S/L | L/N/Q | K | LL/N | L/E | G/Y |
| Mutation 1-52 | T/L | I/Q/Q | R | II/N | V/D | A/Y |

Affinity Analysis

Data is made with an enzyme immunoassay indirect method in the same way of the activity identification, and coating is made into four gradients of 0.5 ug/ml, 0.25 ug/ml, 0.125 ug/ml, and 0.0625 ug/ml; the antibody is diluted by 2 times of the gradient from 1000 ng/ml to 0.195 ng/ml and a sample is loaded. The OD values corresponding to different antibody concentrations with different coating concentrations are obtained. Under the same coating concentration, the antibody concentrations are used as an abscissa, the 00 values are used as an ordinate, and a logarithmic plot is made. According to a fitting equation, the antibody concentration at 50% of the maximum OD value is calculated; it is assigned into a formula: K=(n−1)/(2*(n*Ab′−Ab)), a reciprocal of an affinity constant is calculated, herein Ab and Ab′ respectively represent the antibody concentrations at 50% of the maximum OD value in the corresponding coating concentrations (Ag, Ag′), n=Ag/Ag′; every two coating concentrations may be combined to calculate a K value, and finally six K values may be obtained. An average value thereof is taken and a reciprocal thereof is calculated to get the affinity constant KD.

Affinity Analysis Data of Screened Mutant Antibodies

| | KD(M) |
|---|---|
| Mutation 1 | 5.210E−11 |
| Mutation1-1 | 3.011E−10 |
| Mutation1-2 | 9.031E−11 |
| Mutation1-3 | 3.620E−10 |
| Mutation1-4 | 4.653E−10 |
| Mutation1-5 | 1.251E−10 |
| Mutation1-6 | 4.155E−10 |
| Mutation1-7 | 3.290E−10 |
| Mutation1-8 | 1.982E−10 |
| Mutation 1-9 | 6.451E−11 |
| Mutation1-10 | 8.272E−10 |
| Mutation1-11 | 2.126E−11 |
| Mutation1-12 | 1.259E−10 |
| Mutation1-13 | 8.652E−11 |
| Mutation1-14 | 2.261E−10 |
| Mutation1-15 | 2.452E−10 |
| Mutation1-16 | 5.018E−10 |
| Mutation1-17 | 8.240E−11 |
| Mutation1-18 | 5.096E−11 |
| Mutation1-19 | 8.351E−11 |
| Mutation1-20 | 2.508E−10 |
| Mutation1-21 | 8.285E−11 |
| Mutation1-22 | 8.141E−11 |
| Mutation1-23 | 3.641E−10 |
| Mutation1-24 | 1.268E−10 |
| Mutation1-25 | 4.436E−10 |
| Mutation1-26 | 2.115E−10 |
| Mutation1-27 | 1.647E−10 |
| Mutation1-28 | 1.201E−11 |
| Mutation1-29 | 8.351E−11 |
| Mutation1-30 | 2.328E−10 |
| Mutation1-31 | 9.461E−11 |
| Mutation1-32 | 2.443E−10 |
| Mutation1-33 | 1.689E−10 |
| Mutation1-34 | 1.843E−10 |
| Mutation1-35 | 5.214E−10 |
| Mutation1-36 | 4.726E−10 |
| Mutation1-37 | 3.648E−10 |
| Mutation1-38 | 8.832E−11 |
| Mutation1-39 | 2.334E−10 |
| Mutation1-40 | 8.301E−11 |
| Mutation1-41 | 3.021E−10 |
| Mutation1-42 | 6.426E−11 |
| Mutation1-43 | 1.237E−10 |
| Mutation1-44 | 1.051E−10 |
| Mutation1-45 | 3.837E−10 |
| Mutation1-46 | 7.211E−11 |
| Mutation1-47 | 8.279E−11 |
| Mutation1-48 | 3.328E−10 |
| Mutation1-49 | 9.561E−11 |
| Mutation1-50 | 3.823E−10 |
| Mutation1-51 | 1.787E−10 |
| Mutation1-52 | 1.823E−10 |

Therefore, the mutation sites obtained by screening have a little effect on the affinity of the antibody. In order to verify the above results, the above experiment is repeated by using the WT as the framework sequence, and the affinity of the mutation sites are verified. Some results are as follows.

Mutation Using WT as Framework

| | CDR-VH1 | CDR-VH2 | CDR-VH3 | CDR-VL1 | CDR-VL2 | CDR-VL3 |
|---|---|---|---|---|---|---|
| Site: | X1/X2 | X1/X2/X4 | X1 | X1/X2 | X1/X3 | X1/X2 |
| WT | S/L | L/N/Q | R | II/N | L/D | G/S |
| WT 1-1 | Y/I | V/N/Q | K | LL/N | I/E | A/S |
| WT 1-2 | T/L | I/Q/N | R | IL/Q | L/D | A/T |
| WT 1-3 | S/I | L/N/Q | K | II/N | V/E | G/Y |
| WT 1-4 | T/L | V/N/N | K | LI/Q | I/E | A/T |
| WT 1-5 | Y/I | L/Q/Q | R | LL/Q | L/D | A/S |

Affinity Analysis Data

| | KD(M) |
|---|---|
| WT | 6.334E−10 |
| WT 1-1 | 5.213E−10 |
| WT 1-2 | 8.562E−11 |
| WT 1-3 | 9.232E−11 |
| WT 1-4 | 5.823E−10 |
| WT 1-5 | 3.113E−10 |

It may be seen from the above results that under a precondition of ensuring the antibody activity, the above mutation sites are not closely related to other sites.

The above antibody is paired with another internal antibody (antibody paired with the HRP-II antibody) by the applicant to verify a pairing antibody experiment, and a nature of the antibody is not significantly changed with the WT antibody. It is verified by a double-antibody sandwich pairing experiment, the specificity is maintained in an original high level, and is not significantly changed. It is indicated that the above antibodies are the same epitope as those recognized by the WT antibody before the mutation. Because the overall activity and affinity of Mutation 1 are higher than that of the WT, a detection rate of Mutation 1 corresponding to an application of the kit is also higher than that of the WT, Furthermore, the above antibody specificity may reach 98%-100% in an immunodiagnostic platform test, and the consistency of testing 100 samples reaches 95%-98%.

Furthermore, a stability test is performed on the WT, Mutation 1, and 8 randomly selected mutant antibodies; the above antibodies are stored at 37° C. for 72 hours, and after being taken out, the antibodies stored at 4° C. for 72 hours in the same batch are detected under the same detection condition by the same negative and positive quality control samples. A detection method is the same as the antibody activity analysis method used in the above Embodiments. The linearity of each group of the antibodies may reach more than 99.90%, and a CV value is less than 8%. There is no statistical difference in activity of the antibodies stored at the different temperatures. It is indicated that the above antibodies all have excellent stability, and the mutation of the site has no effect on the stability.

Finally, it should be noted that the above embodiments are only used to explain the technical schemes of the present disclosure, and not to limit them; although the present disclosure is described in detail with reference to the previous embodiments, it should be understood by those of ordinary skill in the art that: it is still possible to modify the technical schemes recorded in each of the previous embodiments, or equivalent replacements are performed on some or all of the technical schemes herein; and these modifications or replacements do not make the essence of the corresponding technical scheme deviate from a scope of the technical schemes of each embodiment of the present disclosure.

### INDUSTRIAL APPLICABILITY

The anti-Plasmodium falciparum HRP-II antibody or the binding protein thereof of the present disclosure is the most recognized diagnostic marker for Plasmodium falciparum, and it may determine the presence of the Plasmodium falciparum in a human body. The detection method for the anti-Plasmodium falciparum HRP-II antibody or the binding protein thereof of the present disclosure is used, and it is simple in operation, fast, and high in sensitivity and specificity.

### Related Sequence Description:

| CDR-VH1. | |
|---|---|
| G-X1-T-X2-T-X3-Y-Y-M-N | (SEQ ID NO. 1) |

herein:

### X1 is Y, T or S;

### X2 is L or I; and

X3 is D or E, preferably D;

CDR-VH2.

| | |
|---|---|
| D-X1-N-P-I-X2-G-G-T-X3-Y-X4-Q-K-F | (SEQ ID NO. 2) |

herein:

### X1 is L, V or I;

### X2 is Q or N;

X3 is A or P, preferably A; and

### X4 is Q or N;

| CDR-VH3. | |
|---|---|
| T-X1-G-A-E-X2-Y | (SEQ ID NO. 3) |

herein:

### X1 is K or R; and

X2 is D or E, preferably D;

CDR-VL1.

| | |
|---|---|
| S-Q-S-X1-Y-S-X2-G-K-I-Y-X3-N | (SEQ ID NO. 4) |

herein:

### X1 is LL, IL, II or LI;

### X2 is Q or N; and

X3 is I or L, preferably L;

| CDR-VL2. | |
|---|---|
| Q-X1-S-K-X2-X3-P | (SEQ ID NO. 5) |

herein:

### X1 is I, V or L;

X2 is I or L, preferably L; and

### X3 is D or E;

| CDR-VL3. | |
|---|---|
| L-Q-X1-T-Y-X2-P-X3-T | (SEQ ID NO. 6) |

herein:

### X1 is A or G;

### X2 is S, Y or T; and

X3 is Q or N, preferably Q;

WT Heavy Chain variable region:
(SEQ ID NO. 35)
EVQLQQSGPEVVKPGASVKISCKASGSTLTEYYMNWVKQSHGKGLEWIGD
LNPINGGTPYQQKFRDKATLTVDKSSNTAYMELRSLTSEDSALYYCTRGA
EEYWGQGTTLTVSS
WT Heavy Chain:
(SEQ ID NO. 36)
EVQLQQSGPEVVKPGASVKISCKASGSTLTEYYMNWVKQSHGKGLEWIGD
LNPINGGTPYQQKFRDKATLTVDKSSNTAYMELRSLTSEDSALYYCTRGA
EEYWGQGTTLTVSSAKTTPPSVYPLAPGCGDTTGSSVTLGCLVKGYFPES
VTVTWNSGSLSSSVHTFPALLQSGLYTMSSSVTVPSSTWPSQTVTCSVAH
PASSTTVDKKLEPSGPISTINPCPPCKECHKCPAPNLEGGPSVFIFPPNI
KDVLMISLTPKVTCVVVDVSEDDPDVQISWFVNNVEVHTAQTQTHREDYN
STIRVVSTLPIQHQDWMSGKEFKCKVNNKDLPSPIERTISKIKGLVRAPQ
VYILPPPAEQLSRKDVSLTCLVVGFNPGDISVEWTSNGHTEENYKDTAPV
LDSDGSYFIYSKLNMKTSKWEKTDSFSCNVRHEGLKNYYLKKTISRSPG
WT Light Chain variable region:
(SEQ ID NO. 37)
DVVLTQTPLSLSVTIGQPASISCKSSQSIIYSNGKIYINWFQQRPGQSPK
RLMYQLSKIDPGIPDRFSGSGSETDFTLKISRVEAEDLGVYYCLQGTYSP
NTFGGGTKLEIK
WT Light Chain:
(SEQ ID NO. 38)
DVVLTQTPLSLSVTIGQPASISCKSSQSIIYSNGKIYINWFQQRPGQSPK
RLMYQLSKIDPGIPDRFSGSGSETDFTLKISRVEAEDLGVYYCLQGTYSP
NTFGGGTKLEIKRADAAPTVSIFPPSSEQLTSGGASVVCFLNNFYPKDIN
VKWKIDGSERQNGVLNSWTDQDSKDSTYSMSSTLTLTKDEYERHNSYTCE
ATHKTSTSPIVKSFNRNEC

### Sequence Description:

| | | SEQ |
|---|---|---|
| Sequence | | ID |
| name | Sequence | NO |
| CDR-VH1 | G-X1-T-X2-T-X3-Y-Y-M-N | 1 |
| CDR-VH2 | D-X1-N-P-I-X2-G-G-T-X3-Y-X4-Q-K-F | 2 |
| CDR-VH3 | T-X1-G-A-E-X2-Y | 3 |
| CDR-VL1 | S-Q-S-X1-Y-S-X2-G-K-I-Y-X3-N | 4 |
| CDR-VL2 | Q-X1-S-K-X2-X3-P | 5 |
| CDR-VL3 | L-Q-X1-T-Y-X2-P-X3-T | 6 |
| SMARTER II A | AAGCAGTGGTATCAACGCAGAGTACXXXXX | 7 |
| Oligonucleotide | | |
| 5′-RACE CDS Primer | (T)25VN | 8 |
| (5′-CDS) | | |
| Universal Primer A Mix | CTAATACGACTCACTATAGGGCAAGCAGTGGTATCAACGCA | 9 |
| (UPM) | GAGT | |
| Nested Universal | AAGCAGTGGTATCAACGCAGAGT | 10 |
| Primer A (NUP) | | |
| mlgG CKR | CGCCTAACACTCATTCCTGTTGAAGC | 11 |
| mlgG CHR | CCGCTCATTTACCCGGAGACCG | 19 |
| Anti-HRP-II3R5-HF | CCCAAGCTTGCCACCATGTACTTGGGACTGAACTGTGTATT | 13 |
| | C | |
| Anti-HRP-II3R5-HR | GGGGAATTCTCATTACTTTCCGGGAGATCTGGAGATGGTTT | 14 |
| | GCTTG | |
| Anti-HRP-II3R5-LF | CCCAAGCTTGCCACCATGGACATCAGGGCTCCTGCTCAGTT | 15 |
| | TCTTGGC | |
| Anti-HRP-II3R5-LR | GGGGAATTCTCATTAACATTCATTTCTGTTAAAAGATTTGACA | 16 |
| | ATG | |
| Variant 1 Heavy Chain | EVQLQQSGPEVVKPGASVKISCKASGSTLTDYYMNWVKQSHG | 17 |
| variable region | KGLEWIGDLNPINGGTAYQQKFRDKATLTVDKSSNTAYMELRS | |
| | LTSEDSALYYCTRGAEDYWGQGTTLTVSS | |
| Variant 1 Heavy Chain | EVQLQQSGPEVVKPGASVKISCKASGSTLTDYYMNWVKQSHG | 18 |
| | KGLEWIGDLNPINGGTAYQQKFRDKATLTVDKSSNTAYMELRS | |
| | LTSEDSALYYCTRGAEDYWGQGTTLTVSSAKTTPPSVYPLAPG | |
| | CGDTTGSSVTLGCLVKGYFPESVTVTWNSGSLSSSVHTFPALL | |
| | QSGLYTMSSSVTVPSSTWPSQTVTCSVAHPASSTTVDKKLEP | |
| | SGPISTINPCPPCKECHKCPAPNLEGGPSVFIFPPNIKDVLMISL | |
| | TPKVTCVVVDVSEDDPDVQISWFVNNVEVHTAQTQTHREDYN | |
| | STIRVVSTLPIQHQDWMSGKEFKCKVNNKDLPSPIERTISKIKGL | |
| | VRAPQVYILPPPAEQLSRKDVSLTCLVVGFNPGDISVEWTSNG | |
| | HTEENYKDTAPVLDSDGSYFIYSKLNMKTSKWEKTDSFSCNVR | |
| | HEGLKNYYLKKTISRSPG | |
| Variant 1 Light Chain | DVVLTQTPLSLSVTIGQPASISCKSSQSIIYSNGKIYLNWFQQRP | 19 |
| variable region | GQSPKRLMYQLSKLDPGIPDRFSGSGSETDFTLKISRVEAEDL | |
| | GVYYCLQGTYSPQTFGGGTKLEIK | |
| Variant 1 Light Chain | DVVLTQTPLSLSVTIGQPASISCKSSQSIIYSNGKIYLNWFQQRP | 20 |
| | GQSPKRLMYQLSKLDPGIPDRFSGSGSETDFTLKISRVEAEDL | |
| | GVYYCLQGTYSPQTFGGGTKLEIKRADAAPTVSIFPPSSEQLTS | |
| | GGASVVCFLNNFYPKDINVKWKIDGSERQNGVLNSWTDQDSK | |
| | DSTYSMSSTLTLTKDEYERHNSYTCEATHKTSTSPIVKSFNRN | |
| | EC | |
| Variant 1 HEAVY | GSTLTDYYMN | 21 |
| CHAIN CDR1 | | |
| Variant 1 HEAVY | DLNPINGGTAYQQKF | 22 |
| CHAIN CDR2 | | |
| Variant 1 HEAVY | TRGAEDY | 23 |
| CHAIN CDR3 | | |
| Variant 1 LIGHT CHAIN | SQSIIYSNGKIYLN | 24 |
| CDR1 | | |
| Variant 1 LIGHT CHAIN | QLSKLDP | 25 |
| CDR2 | | |
| Variant 1 LIGHT CHAIN | LQGTYSPQT | 26 |
| CDR3 | | |
| FR-H1 | EVQLQQSGPEVVKPGASVKISCKAS | 27 |
| FR-H2 | WVKQSHGKGLEWIG | 28 |
| FR-H3 | RDKATLTVDKSSNTAYMELRSLTSEDSALYYC | 29 |
| FR-H4 | WGQGTTLTVSS | 30 |
| FR-L1 | DVVLTQTPLSLSVTIGQPASISCKS | 31 |
| FR-L2 | WFQQRPGQSPKRLMY | 32 |
| FR-L3 | GIPDRFSGSGSETDFTLKISRVEAEDLGVYYC | 33 |
| FR-L4 | FGGGTKLEIK | 34 |

What is claimed is:

1. An antibody or an antigen-binding fragment thereof, wherein the antibody binds to a Plasmodium falciparum histidine-rich protein-II, namely HRP-II, wherein the antibody comprises complementarity determining regions having the following amino acid sequences or at least 80% sequence identity with thereof:
a heavy chain CDR1, which comprises or consists of an amino acid sequence G-X1-T-X2-T-X3-Y-Y-M-N shown in SEQ ID NO:1, wherein X1 is Y, T or S; X2 is L or I; and X3 is D or E, preferably D;
a heavy chain CDR2, which comprises or consists of an amino acid sequence D-X1-N-P-I-X2-G-G-T-X3-Y-X4-Q-K-F shown in SEQ ID NO: 2, wherein X1 is L, V or I; X2 is Q or N; X3 is A or P, preferably A; and X4 is Q or N; and
a heavy chain CDR3, which comprises or consists of an amino acid sequence T-X1-G-A-E-X2-Y shown in SEQ ID NO: 3, wherein X1 is K or R; and X2 is D or E, preferably D; and
the antibody further comprises:
a light chain CDR1, which comprises or consists of an amino acid sequence S-Q-S-X1-Y-S-X2-G-K-Y-X3-N shown in SEQ ID NO: 4, wherein X1 is LL, IL, II or LI; X2 is Q or N; and X3 is I or L, preferably L;
a light chain CDR2, which comprises or consists of an amino acid sequence Q-X1-S-K-X2-X3-P shown in SEQ ID NO: 5, wherein X1 is I, V or L; X2 is I or L, preferably L; and X3 is D or E; and
a light chain CDR3, which comprises or consists of an amino acid sequence L-Q-X1-T-Y-X2-P-X3-T shown in SEQ ID NO: 6, wherein X1 is A or G; X2 is S, Y or T; and X3 is Q or N, preferably Q.

2. The antibody or the antigen-binding fragment thereof as claimed in claim 1, wherein the antibody comprises:
a heavy chain CDR1, which comprises or consists of an amino acid sequence shown in SEQ ID NO: 21;
a heavy chain CDR2, which comprises or consists of an amino acid sequence shown in SEQ ID NO: 22; and
a heavy chain CDR3, which comprises or consists of an amino acid sequence shown in SEQ ID NO: 23; and
the antibody further comprises:
a light chain CDR1, which comprises or consists of an amino acid sequence shown in SEQ ID NO: 24;
a light chain CDR2, which comprises or consists of an amino acid sequence shown in SEQ ID NO: 25; and
a light chain CDR3, which comprises or consists of an sequence shown in SEQ ID NO: 26.

3. The antibody or the antigen-binding fragment thereof as claimed in claim 1, wherein the antibody comprises:
(i) a heavy chain variable region, which comprises or consists of the following sequences:
an amino acid sequence shown in SEQ ID NO: 17, or
a sequence having at least 70%, 75%, 80%, 85%, and 90%, preferably at least 91%, 92%, 93%, 94%, 95%, 96%, 97%, 98%, 99% or 100%, of sequence identity with the sequence shown in the SEQ ID NO: 17, or
an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 17; and
(II) a light chain variable region, which comprises or consists of the following sequences:
an amino acid sequence shown in SEQ ID NO: 19, or
a sequence having at least 70%, 75%, 80%, 85%, and 90%, preferably at least 91%, 92%, 93%, 94%, 95%, 96%, 97%, 98%, 99% or 100%, of sequence identity with the sequence shown in the SEQ ID NO: 19, or
an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 19.

4. The antibody or the antigen-binding fragment thereof as claimed in claim 1, wherein the antibody binds to the HRP-II protein in a KD of 10⁻¹⁰ M or less.

5. The antibody or the antigen-binding fragment thereof as claimed in claim 1, wherein the antibody binds to the HRP-II protein in an EC50 of less than about 100 nM, for example, less than about 10 nM, 1 nM, 0.9 nM, 0.8 nM, 0.7 nM, 0.6 nM, 0.5 nM, 0.4 nM, 0.3 nM, 0.2 nM, 0.1 nM or less.

6. The antibody or the antigen-binding fragment thereof as claimed in claim 1, wherein the antigen-binding fragment is selected from Fab, Fab′, F(ab′)₂, Fd, Fv, a complementarity determining region (CDR) fragment, a single-chain antibody (for example, scFv), a bivalent antibody or a domain antibody.

7. An isolated polypeptide, which is selected from the group consisting of the following items:
(1) an isolated polypeptide, which comprises sequences shown in SEQ ID NO: 21, 22 and 23, wherein the polypeptide is served as a part of an anti-HRP-II antibody, and specifically binds to the HRP-II, and the antibody further comprises sequences shown in SEQ ID NO: 24, 25 and 26;
(2) an isolated polypeptide, which comprises sequences shown in SEQ ID NO: 24, 25 and 26, wherein the polypeptide is served as a part of the anti-HRP-II antibody, and specifically binds to the HRP-II, and the antibody further comprises sequences shown in SEQ ID NOs: 21, 22 and 23;
(3) an isolated polypeptide, which comprises the sequence shown in the SEQ ID NO: 17, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, wherein the polypeptide is served as a part of the anti-HRP-II antibody, and specifically binds to the HRP-II, and the antibody further comprises the sequence shown in the SEQ ID NO: 19, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence;
(4) an isolated polypeptide, which comprises the sequence shown in the SEQ ID NO: 19, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, wherein the polypeptide is served as a part of the anti-HRP-II antibody, and specifically binds to the HRP-II, and the antibody further comprises the sequence shown in the SEQ ID NO: 17, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence;
(5) an isolated polypeptide, which comprises the sequence shown in the SEQ ID NO: 18, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, wherein the polypeptide is served as a part of the anti-HRP-II antibody, and specifically binds to the HRP-II, and the antibody further comprises the sequence shown in the SEQ ID NO: 20, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence; and
(6) an isolated polypeptide, which comprises the sequence shown in the SEQ ID NO: 20, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence, wherein the polypeptide is served as a part of the anti-HRP-II antibody, and specifically binds to the HRP-II, and the antibody further comprises the sequence shown in the SEQ ID NO: 18, or the sequence having at least 70%, 75%, 80%, 85%, and 90%, preferably at least 91%, 92%, 93%, 94%, 95%, 96%, 97%, 98%, 99% or 100%, of sequence identity with the sequence, or the amino acid sequence having one or more (preferably 1, 2, 3, 4, 5, 6, 7, 8, 9 or 10) amino acid mutations (preferably a conservative mutation, preferably substitution, insertion or deletion) compared with the sequence.

8. An isolated polynucleotide, which encodes the antibody or the antigen-binding fragment thereof as claimed in claim 1.

9. A vector, which comprises the isolated polynucleotide as claimed in claim 8.

10. A host cell, which comprises the isolated polynucleotide as claimed in claim 8.

11. A method for preparing the antibody or the antigen-binding fragment thereof as claimed in claim 1, wherein the method comprises culturing a host cell wherein the host cell comprises an isolated polynucleotide encoding the antibody or the antigen-binding fragment thereof as claimed in claim 1.

12. An antibody conjugate, which comprises the antibody or the antigen-binding fragment thereof as claimed in claim 1 and a coupling portion coupled with the same, preferably, the coupling portion comprises a purification tag (such as a His tag), a detectable label, such as a colloidal gold, a radioactive label, a luminescent substance, a colored substance, an enzyme, such as a fluorescent label, a chromophore label, an electronic dense label, such as a radioisotope, a fluorophore, rhodamine and a derivative thereof, a luciferase, a luciferin, a horseradish peroxidase, an alkaline phosphatase, a p-galactosidase, a glucoamylase, a lysozyme, a carbohydrate oxidase, a glucose oxidase, a galactose oxidase, and a glucose-6-phosphate dehydrogenase, biotin/avidin, and a spin label.

13. A kit or a diagnostic agent, which comprises the antibody or the antigen-binding fragment thereof as claimed in claim 1, wherein:
1) preferably, the kit or the diagnostic agent further comprises an antibody which binds to a Plasmodium antigen except the HRP-II or an antigen-binding fragment thereof, an antibody conjugate, or a fusion protein, the Plasmodium antigen except the HRP-II comprises, for example, Plasmodium falciparum, Plasmodium vivax, Plasmodium malariae and/or Plasmodium ovale specific antigens or shared antigens, such as an aldolase, such as a lactate dehydrogenase PLDH, such as a Plasmodium lactate dehydrogenase PLDH or a Plasmodium species-specific lactate dehydrogenase PLDH, such as a Plasmodium glutamate dehydrogenase PGDH, and Plasmodium antigens LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP;
2) preferably, the kit or the diagnostic agent further comprises another one or more antibodies, which specifically recognizes the antibody or the antigen-binding fragment as claimed in claim 1; and/or specifically recognizes the antibody which binds to the Plasmodium antigen except the HRP-II or the antigen-binding fragment thereof, the antibody conjugate, or the fusion protein, optionally, the another one or more antibodies further comprise a detectable label, such as a colloidal gold, a radioactive label, a luminescent substance, a colored substance, an enzyme, such as a fluorescent label, a chromophore label, an electronic dense label, such as a radioisotope, a fluorophore, rhodamine and a derivative thereof, a luciferase, a luciferin, a horseradish peroxidase, an alkaline phosphatase, a β-galactosidase, a glucoamylase, a lysozyme, a carbohydrate oxidase, a glucose oxidase, a galactose oxidase, and a glucose-6-phosphate dehydrogenase, biotin/avidin, and a spin label;
3) optionally, the kit is a Rapid Diagnostic Test (RDT) kit, such as a kit for applying thin film immunochromatography, for example, a kit for thin film immunochromatography using a colloidal gold method, for example, a solid-phase support such as a membrane support is contained, the antibody which binds to the Plasmodium antigen or the antigen-binding fragment thereof, the antibody conjugate, or the fusion protein and an optional control antibody are immobilized to the solid-phase support; and
4) optionally, the kit is an Enzyme-Linked ImmunoSorbent Assay (ELISA) kit, an indirect ImmunoFluorescence Assay (IFA) kit, and a RadioImmuno Assay (RIA) kit.

14. The diagnostic agent or the kit as claimed in claim 13, wherein the diagnostic agent or the kit is used for 1) detecting the presence of the HRP-II in a sample or a level thereof, 2) diagnosing a Plasmodium falciparum infection, and/or 3) identifying and diagnosing the Plasmodium falciparum infection and other Plasmodium infections, optionally the sample comprises a tissue, a cell or a fluid sample, such as a sample of a body fluid, such as a cerebrospinal fluid, a urine, a saliva, a blood sample.

15. A diagnosis or detection method, wherein the method comprises contacting a biological sample with the antibody or the antigen-binding fragment thereof as claimed in claim 1 under a condition that the antibody is allowed to bind with a target, and detecting whether a complex is formed between the antibody and the target, the method is used for 1) detecting the presence of the HRP-II in a sample or a level thereof, 2) diagnosing a Plasmodium falciparum infection, and/or 3) identifying and diagnosing the Plasmodium falciparum infection and other Plasmodium infections, optionally the sample comprises a tissue, a cell or a fluid sample, such as a sample of a body fluid, such as a cerebrospinal fluid, a urine, a saliva, a blood sample.

16. The antibody or the antigen-binding fragment thereof as claimed in claim 1, wherein the antibody comprises the heavy chain CDR1, the heavy chain CDR2, the heavy chain CDR3, the light chain CDR1, the light chain CDR2, and the light chain CDR3 containing amino acid residue substitution combinations as shown in the following table:
i) the antibody comprises the amino acid residue substitution combinations shown in the table below:

| | CDR-VH1 | CDR-VH2 | CDR-VH3 | CDR-VL1 | CDR-VL2 | CDR-VL3 |
|---|---|---|---|---|---|---|
| Site | X3 | X3 | X2 | X3 | X2 | X3 |
| Mutation 1 | D | A | D | L | L | Q |

ii) the antibody in the i) further comprises the amino acid residue substitution combinations shown in the table below:

| | CDR-VH1 | CDR-VH2 | CDR-VH3 | CDR-VL1 | CDR-VL2 | CDR-VL3 |
|---|---|---|---|---|---|---|
| Site | X1/X2 | X1/X2/X4 | X1 | X1/X2 | X1/X3 | X1/X2 |
| Mutation 1-0 | Y/L | L/Q/N | K | LL/Q | I/D | A/S |
| Mutation 1-1 | Y/I | I/Q/N | R | II/Q | l/E | G/Y |
| Mutation 1-2 | S/L | V/Q/Q | K | LL/N | L/D | A/T |
| Mutation 1-3 | Y/I | L/N/Q | R | II/N | L/E | G/S |
| Mutation 1-4 | Y/L | I/N/Q | K | IL/N | V/D | A/Y |
| Mutation 1-5 | S/L | V/N/N | R | IL/Q | V/E | G/T |
| Mutation 1-6 | Y/I | L/Q/N | K | LI/N | l/D | A/S |
| Mutation 1-7 | Y/I | I/N/Q | R | LI/N | I/E | A/Y |
| Mutation 1-8 | Y/I | V/Q/N | K | LL/Q | L/D | A/T |
| Mutation 1-9 | Y/L | L/Q/N | R | II/Q | L/E | G/S |
| Mutation 1-10 | Y/I | I/N/N | R | LL/N | V/D | G/Y |
| Mutation 1-11 | Y/I | V/N/N | K | II/N | V/E | A/T |
| Mutation 1-12 | Y/I | L/N/N | R | IL/N | I/D | G/S |
| Mutation 1-13 | S/L | I/Q/N | K | IL/Q | I/E | G/Y |
| Mutation 1-14 | Y/L | V/N/N | K | LI/N | L/D | A/T |
| Mutation 1-15 | Y/I | L/Q/N | R | LI/N | L/E | G/T |
| Mutation 1-16 | Y/I | I/Q/N | R | LL/Q | V/D | G/S |
| Mutation 1-17 | Y/I | V/N/N | K | LL/Q | V/E | G/Y |
| Mutation 1-18 | Y/L | L/Q/N | K | LL/Q | I/D | G/T |
| Mutation 1-19 | T/L | I/Q/N | R | II/Q | I/E | A/S |
| Mutation 1-20 | S/I | V/Q/N | K | II/Q | L/D | G/Y |
| Mutation 1-21 | T/L | L/Q/N | R | LL/Q | L/E | G/T |
| Mutation 1-22 | Y/I | I/Q/Q | K | II/N | V/D | A/S |
| Mutation 1-23 | T/L | V/Q/N | R | II/N | V/E | G/Y |
| Mutation 1-24 | T/L | L/Q/Q | K | II/N | I/D | A/S |
| Mutation 1-25 | Y/I | I/Q/N | R | LL/Q | I/E | G/T |
| Mutation 1-26 | T/L | V/Q/N | R | LL/Q | L/D | G/Y |
| Mutation 1-27 | T/L | L/N/Q | K | II/N | L/E | G/S |
| Mutation 1-28 | Y/I | I/Q/Q | K | II/N | V/D | A/T |
| Mutation 1-29 | Y/I | V/Q/N | R | II/Q | V/E | G/Y |
| Mutation 1-30 | S/L | L/Q/N | K | II/N | I/D | A/S |
| Mutation 1-31 | T/L | I/N/Q | K | LL/N | I/E | G/T |
| Mutation 1-32 | Y/I | V/Q/Q | R | LL/N | L/D | A/Y |
| Mutation 1-33 | T/L | L/Q/N | K | LL/N | L/E | G/S |
| Mutation 1-34 | T/L | I/N/Q | R | LL/N | V/D | G/T |
| Mutation 1-35 | Y/I | V/Q/Q | R | LL/N | V/E | A/Y |
| Mutation 1-36 | T/L | L/Q/N | R | LL/N | I/D | G/S |
| Mutation 1-37 | Y/I | I/N/Q | K | LL/N | I/E | A/Y |
| Mutation 1-38 | T/L | V/Q/Q | R | LL/N | L/D | G/S |
| Mutation 1-39 | T/L | L/N/Q | K | LL/N | L/E | A/T |
| Mutation 1-40 | T/L | I/N/Q | R | IL/N | L/D | G/T |
| Mutation 1-41 | Y/L | V/N/Q | K | IL/N | V/E | A/Y |
| Mutation 1-42 | Y/I | L/N/Q | R | IL/N | I/D | G/S |
| Mutation 1-43 | T/L | I/N/Q | K | IL/N | I/E | G/S |
| Mutation 1-44 | Y/I | V/N/Q | R | IL/N | L/D | A/S |
| Mutation 1-45 | Y/I | L/N/N | K | IL/Q | L/E | G/Y |
| Mutation 1-46 | Y/I | I/N/N | R | IL/Q | V/D | G/T |
| Mutation 1-47 | S/I | V/N/N | K | IL/Q | V/E | A/S |
| Mutation 1-48 | Y/I | L/Q/N | R | LL/Q | I/D | G/Y |
| Mutation 1-49 | Y/L | I/Q/N | K | LL/Q | I/E | A/T |
| Mutation 1-50 | Y/I | V/Q/Q | R | LL/N | L/E | A/Y |
| Mutation 1-51 | S/L | L/N/Q | K | LL/N | L/E | G/Y |
| Mutation 1-52 | T/L | I/Q/Q | R | II/N | V/D | A/Y; |

optionally, wherein the antibody binds to the HRP-II protein in a KD of 10⁻⁹M or less.

17. The antibody or the antigen-binding fragment thereof as claimed in claim 1, wherein the antibody or the antigen-binding fragment thereof is immobilized on a surface;
preferably, wherein the surface is a solid-phase support, a membrane, a glass or a metal support;
preferably, wherein the solid-phase support is a plastic;
preferably, wherein the membrane is a nitrocellulose membrane.

18. The antibody or the antigen-binding fragment thereof as claimed in claim 2, wherein the antibody further comprises framework regions of a heavy chain variable region FR-H1, FR-H2, FR-H3 and FR-H4 and framework regions of a light chain variable region FR-L1, FR-L2, FR-L3 and FR-L4,
wherein the FR-H1 comprises or consists of an amino acid sequence of SEQ ID NO: 27, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 27, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 27;
the FR-H2 comprises or consists of an amino acid sequence of SEQ ID NO: 28, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 28, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 28;
the FR-H3 comprises or consists of an amino acid sequence of SEQ ID NO: 29, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 29, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 29;
the FR-H4 comprises or consists of an amino acid sequence of SEQ ID NO: 30, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 30, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 30;
the FR-L1 comprises or consists of an amino acid sequence of SEQ ID NO: 31, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 31, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 31;
the FR-L2 comprises or consists of an amino acid sequence of SEQ ID NO: 32, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 32, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 32;
the FR-L3 comprises or consists of an amino acid sequence of SEQ ID NO: 33, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 33, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 33; and
the FR-L4 comprises or consists of an amino acid sequence of SEQ ID NO: 34, or a sequence having at least 70%, 75%, 80%, 85%, 90%, preferably at least 91%, 92%, 93% 94%, 95%, 96%, 97%, 98%, 99% or 100%, of sequence identity with the sequence shown in the SEQ ID NO: 34, or an amino acid sequence having one or more (preferably 1, 2, 3, 4, 5, 6, 7, 8, 9 or 10) amino acid mutations (preferably a conservative mutation, preferably substitution, insertion or deletion) compared with the amino acid sequence shown in the SEQ ID NO: 34.

19. A kit or a diagnostic agent, which comprises the antibody conjugate as claimed in claim 13, wherein:
1) preferably, the kit or the diagnostic agent further comprises an antibody which binds to a Plasmodium antigen except the HRP-II or an antigen-binding fragment thereof, an antibody conjugate, or a fusion protein, the Plasmodium antigen except the HRP-II comprises, for example, Plasmodium falciparum, Plasmodium vivax, Plasmodium malariae and/or Plasmodium ovale specific antigens or shared antigens, such as an aldolase, such as a lactate dehydrogenase PLDH, such as a Plasmodium lactate dehydrogenase PLDH or a Plasmodium species-specific lactate dehydrogenase PLDH, such as a Plasmodium glutamate dehydrogenase PGDH, and Plasmodium antigens LSA-1, LSA-3, LSA-5, SALSA, STARP, TRAP, PfEXP1, CS, MSP-3-1, MSP-3-2, MSP-3-5, MSP-3-6, MSP1, MSP2, MSP4, MSP5, AMA-1, SERP and GLURP;
2) preferably, the kit or the diagnostic agent further comprises another one or more antibodies, which specifically recognizes the antibody or the antigen-binding fragment as claimed in claim 1; and/or specifically recognizes the antibody which binds to the Plasmodium antigen except the HRP-II or the antigen-binding fragment thereof, the antibody conjugate, or the fusion protein, optionally, the another one or more antibodies further comprise a detectable label, such as a colloidal gold, a radioactive label, a luminescent substance, a colored substance, an enzyme, such as a fluorescent label, a chromophore label, an electronic dense label, such as a radioisotope, a fluorophore, rhodamine and a derivative thereof, a luciferase, a luciferin, a horseradish peroxidase, an alkaline phosphatase, a P-galactosidase, a glucoamylase, a lysozyme, a carbohydrate oxidase, a glucose oxidase, a galactose oxidase, and a glucose-6-phosphate dehydrogenase, biotin/avidin, and a spin label;
3) optionally, the kit is a Rapid Diagnostic Test (RDT) kit, such as a kit for applying thin film immunochromatography, for example, a kit for thin film immunochromatography using a colloidal gold method, for example, a solid-phase support such as a membrane support is contained, the antibody which binds to the Plasmodium antigen or the antigen-binding fragment thereof, the antibody conjugate, or the fusion protein and an optional control antibody are immobilized to the solid-phase support; and
4) optionally, the kit is an Enzyme-Linked ImmunoSorbent Assay (ELISA) kit, an indirect ImmunoFluorescence Assay (IFA) kit, and a RadioImmuno Assay (RIA) kit.

20. The diagnostic agent or the kit as claimed in claim 19, wherein the diagnostic agent or the kit is used for 1) detecting the presence of the HRP-II in a sample or a level thereof, 2) diagnosing a Plasmodium falciparum infection, and/or 3) identifying and diagnosing the Plasmodium falciparum infection and other Plasmodium infections, optionally the sample comprises a tissue, a cell or a fluid sample, such as a sample of a body fluid, such as a cerebrospinal fluid, a urine, a saliva, a blood sample.


SEQUENCE LISTING (38 sequences)

SEQ ID NO: 1 - protein, 10 aa, Artificial
  CDR-VH1
  misc_feature (2)..(2): Xaa can be Tyr, Thr or Ser
  misc_feature (4)..(4): Xaa can be Leu or Ile
  misc_feature (6)..(6): Xaa can be Asp or Glu, preferably Asp
GXTXTXYYMN

SEQ ID NO: 2 - protein, 15 aa, Artificial
  CDR-VH2
  misc_feature (2)..(2): Xaa can be Leu, Val or Ile
  misc_feature (6)..(6): Xaa can be Gln or Asn
  misc_feature (10)..(10): Xaa can be Ala or Pro, preferably Ala
  misc_feature (12)..(12): Xaa can be Gln or Asn
DXNPIXGGTXYXQKF

SEQ ID NO: 3 - protein, 7 aa, Artificial
  CDR-VH3
  misc_feature (2)..(2): Xaa can be Lys or Arg
  misc_feature (6)..(6): Xaa can be Asp or Glu, preferably Asp
TXGAEXY

SEQ ID NO: 4 - protein, 14 aa, Artificial
  CDR-VL1
  misc_feature (4)..(4): Xaa can be Leu or Ile
  misc_feature (5)..(5): Xaa can be Leu or Ile
  misc_feature (8)..(8): Xaa can be Gln or Asn
  misc_feature (13)..(13): Xaa can be Ile or Leu, preferably Leu
SQSXXYSXGKIYXN

SEQ ID NO: 5 - protein, 7 aa, Artificial
  CDR-VL2
  misc_feature (2)..(2): Xaa can be Ile, Val or Leu
  misc_feature (5)..(5): Xaa can be Ile or Leu, preferably Leu
  misc_feature (6)..(6): Xaa can be Asp or Glu
QXSKXXP

SEQ ID NO: 6 - protein, 9 aa, Artificial
  CDR-VL3
  misc_feature (3)..(3): Xaa can be Ala or Gly
  misc_feature (6)..(6): Xaa can be Ser, Tyr or Thr
  misc_feature (8)..(8): Xaa can be Gln or Asn, preferably Gln
LQXTYXPXT

SEQ ID NO: 7 - DNA, 30 bp, Artificial
  SMARTER II A Oligonucleotide
  misc_feature (26)..(30): n is a, c, g, or t
aagcagtggtatcaacgcagagtacnnnnn

SEQ ID NO: 8 - DNA, 27 bp, Artificial
  5′RACE CDS Primer5′CDS)
  misc_feature (26)..(26): v is a or g or c
  misc_feature (27)..(27): n is a, c, g, or t
tttttttttttttttttttttttttvn

SEQ ID NO: 9 - DNA, 45 bp, Artificial
  Universal Primer A Mix(UPM)
ctaatacgactcactatagggcaagcagtggtatcaacgcagagt

SEQ ID NO: 10 - DNA, 23 bp, Artificial
  Nested Universal Primer A(NUP)
aagcagtggtatcaacgcagagt

SEQ ID NO: 11 - DNA, 26 bp, Artificial
  mIgG CKR
cgcctaacactcattcctgttgaagc

SEQ ID NO: 12 - DNA, 22 bp, Artificial
  mIgG CHR
ccgctcatttacccggagaccg

SEQ ID NO: 13 - DNA, 42 bp, Artificial
  Anti HRPII3R5HF
cccaagcttgccaccatgtacttgggactgaactgtgtattc

SEQ ID NO: 14 - DNA, 46 bp, Artificial
  Anti HRPII3R5HR
ggggaattctcattactttccgggagatctggagatggtttgcttg

SEQ ID NO: 15 - DNA, 48 bp, Artificial
  Anti HRPII3R5LF
cccaagcttgccaccatggacatcagggctcctgctcagtttcttggc

SEQ ID NO: 16 - DNA, 46 bp, Artificial
  Anti HRPII3R5LR
ggggaattctcattaacattcatttctgttaaaagatttgacaatg

SEQ ID NO: 17 - protein, 114 aa, Artificial
  Variant 1 Heavy Chain variable region
EVQLQQSGPEVVKPGASVKISCKASGSTLTDYYMNWVKQSHGKGLEWIGDLNPINGGTAYQQKFRDKATLTVDKSSNTAYMELRSLTSEDSALYYCTRGAEDYWGQGTTLTVSS

SEQ ID NO: 18 - protein, 449 aa, Artificial
  Variant 1 Heavy Chain
EVQLQQSGPEVVKPGASVKISCKASGSTLTDYYMNWVKQSHGKGLEWIGDLNPINGGTAYQQKFRDKATLTVDKSSNTAYMELRSLTSEDSALYYCTRGAEDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 19 - protein, 112 aa, Artificial
  Variant 1 Light Chain variable region
DVVLTQTPLSLSVTIGQPASISCKSSQSIIYSNGKIYLNWFQQRPGQSPKRLMYQLSKLDPGIPDRFSGSGSETDFTLKISRVEAEDLGVYYCLQGTYSPQTFGGGTKLEIK

SEQ ID NO: 20 - protein, 219 aa, Artificial
  Variant 1 Light Chain
DVVLTQTPLSLSVTIGQPASISCKSSQSIIYSNGKIYLNWFQQRPGQSPKRLMYQLSKLDPGIPDRFSGSGSETDFTLKISRVEAEDLGVYYCLQGTYSPQTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 10 aa, Artificial
  Variant 1 HEAVY CHAIN CDR1
GSTLTDYYMN

SEQ ID NO: 22 - protein, 15 aa, Artificial
  Variant 1 HEAVY CHAIN CDR2
DLNPINGGTAYQQKF

SEQ ID NO: 23 - protein, 7 aa, Artificial
  Variant 1 HEAVY CHAIN CDR3
TRGAEDY

SEQ ID NO: 24 - protein, 14 aa, Artificial
  Variant 1 LIGHT CHAIN CDR1
SQSIIYSNGKIYLN

SEQ ID NO: 25 - protein, 7 aa, Artificial
  Variant 1 LIGHT CHAIN CDR2
QLSKLDP

SEQ ID NO: 26 - protein, 9 aa, Artificial
  Variant 1 LIGHT CHAIN CDR3
LQGTYSPQT

SEQ ID NO: 27 - protein, 25 aa, Artificial
  FR-H1
EVQLQQSGPEVVKPGASVKISCKAS

SEQ ID NO: 28 - protein, 14 aa, Artificial
  FR-H2
WVKQSHGKGLEWIG

SEQ ID NO: 29 - protein, 32 aa, Artificial
  FR-H3
RDKATLTVDKSSNTAYMELRSLTSEDSALYYC

SEQ ID NO: 30 - protein, 11 aa, Artificial
  FR-H4
WGQGTTLTVSS

SEQ ID NO: 31 - protein, 25 aa, Artificial
  FR-L1
DVVLTQTPLSLSVTIGQPASISCKS

SEQ ID NO: 32 - protein, 15 aa, Artificial
  FR-L2
WFQQRPGQSPKRLMY

SEQ ID NO: 33 - protein, 32 aa, Artificial
  FR-L3
GIPDRFSGSGSETDFTLKISRVEAEDLGVYYC

SEQ ID NO: 34 - protein, 10 aa, Artificial
  FR-L4
FGGGTKLEIK

SEQ ID NO: 35 - protein, 114 aa, Artificial
  WT Heavy Chain variable region
EVQLQQSGPEVVKPGASVKISCKASGSTLTEYYMNWVKQSHGKGLEWIGDLNPINGGTPYQQKFRDKATLTVDKSSNTAYMELRSLTSEDSALYYCTRGAEEYWGQGTTLTVSS

SEQ ID NO: 36 - protein, 449 aa, Artificial
  WT Heavy Chain
EVQLQQSGPEVVKPGASVKISCKASGSTLTEYYMNWVKQSHGKGLEWIGDLNPINGGTPYQQKFRDKATLTVDKSSNTAYMELRSLTSEDSALYYCTRGAEEYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 37 - protein, 112 aa, Artificial
  WT Light Chain variable region
DVVLTQTPLSLSVTIGQPASISCKSSQSIIYSNGKIYINWFQQRPGQSPKRLMYQLSKIDPGIPDRFSGSGSETDFTLKISRVEAEDLGVYYCLQGTYSPNTFGGGTKLEIK

SEQ ID NO: 38 - protein, 219 aa, Artificial
  WT Light Chain
DVVLTQTPLSLSVTIGQPASISCKSSQSIIYSNGKIYINWFQQRPGQSPKRLMYQLSKIDPGIPDRFSGSGSETDFTLKISRVEAEDLGVYYCLQGTYSPNTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC